(19) United States
(12) Patent Application Publication
(10) Pub. No.: US 2019/0015369 A1
(43) Pub. Date: Jan. 17, 2019

(54) METHOD OF ACNE TREATMENT BY CONCOMITANT TOPICAL ADMINISTRATION OF BENZOYL PEROXIDE AND TRETINOIN

(71) Applicant: SOL-GEL TECHNOLOGIES LTD., Ness Ziona (IL)

(72) Inventors: Moshe Arkin, Kfar Shmaryahu (IL); Stephen Cherkez, Caesarea (IL); Ofer Toledano, Kfar Saba (IL)

(73) Assignee: SOL-GEL TECHNOLOGIES LTD., Ness Ziona (IL)

(21) Appl. No.: 15/978,146

(22) Filed: May 13, 2018

Related U.S. Application Data
(63) Continuation of application No. 15/647,331, filed on Jul. 12, 2017.

(51) Int. Cl. A61K 31/203; A61K 47/14; A61K 9/50; A61K 31/327; A61K 9/00

(52) U.S. Cl. CPC A61K 31/203; A61K 47/14; A61K 9/501; A61K 9/0014; A61K 31/327

(57) ABSTRACT

The present disclosure relates to methods of treatment of acne in a patient in need thereof, comprising the concomitant once daily topical administration of from about 20 w/w to about 10% w/w of solid particulate benzoyl peroxide (BPO) and from about 0.01% w/w to about 0.1% w/w of solid particulate all trans retinoic acid (ATRA) for a period of up to 3 months, wherein the side-effects of the two actives are medically acceptable and wherein the therapeutic effect of BPO and ATRA is superior to the effect of each active administered alone. The concomitant administration may be carried out by once daily administration to a patient in need thereof of a single composition or of a first composition comprising BPO and a second composition comprising ATRA from a dual chamber dispenser or from two separate dispensers, mixed before applying on the skin of a patient in need thereof for up to 2 weeks, up to 1 month, preferably up to 2 months and more preferably up to 3 months.

### CROSS-REFERENCE TO RELATED APPLICATIONS

This application is a continuation of U.S. patent application Ser. No. 15/647,331, filed Jul. 12, 2017, which is hereby incorporated by reference.

### FIELD OF THE INVENTION

The present invention relates to methods of treatment of acne in a patient in need thereof, comprising the concomitant once daily topical administration of solid particulate benzoyl peroxide (BPO) and solid particulate all trans retinoic acid (tretinoin or ATRA) for a period of up to 3 months.

The concomitant once daily topical administration may be done by once daily topical administration of a single composition comprising both BPO and ATRA, or alternatively, by topical administration of a first composition comprising BPO and a second composition comprising ATRA, concomitantly administered once daily from a dual chamber dispenser or from two separate dispensers and mixed before applying on the skin of a patient in need thereof.

### BACKGROUND

### Description of the Related Art

Methods of Acne Treatment with Tretinoin/BPO

Clinicians have been reluctant to prescribe topical retinoids and BPO concurrently due to a belief that the BPO may result in oxidation and degradation of the tretinoin molecule, thereby reducing its effectiveness, and prefer to recommend the BPO or an antibiotic/BPO combination to be applied in the morning and tretinoin at night (Yan A C. Current concepts in acne management. Adolesc. Med. Clin. 2006; 17(3):613-637.)

Another publication (Emmy Graber, Treatment of Acne Vulgaris, UpToDate.com, July 2016) states “topical tretinoin should NOT be applied at the same time as benzoyl peroxide”, despite the known fact that newer retinoid compositions like Retin A microspheres (MICROSPONGE® System) have less interaction or no short term interaction with BPO. Obviously, concomitant administration of tretinoin and BPO is taught away by this publication.

Unlike adapalene, which is often combined with BPO, tretinoin is significantly more irritant to the skin and since BPO is also irritant, it has been feared that the two APIs together will create unacceptable cutaneous side effects. Also, BPO is known to oxidize tretinoin and hence it was feared that their interaction on the skin when administered together will diminish the therapeutic effect of tretinoin. Thus, while there are some reports in the literature on the value of both compounds being administered one in the morning and the other in the evening, the verdict up to now was that the two products should not be administered concomitantly.

This belief of the medical profession explains why all previous attempts to solve the stability problem of tretinoin/BPO, such as microencapsulation technology, did not yield a commercial product so far.

### Compositions

Microcapsules having a core surrounded within a metal oxide shell have been proposed as controlled forms of topically administered compositions. It has been shown that various active agents, such as anti-acne agents, can be encapsulated within the metal oxide shell. For example, International Application No. PCT/IL/2008/000140 (published as WO 2008/093346) and U.S. Pat. No. 8,617,580 describe methods and compositions comprising benzoyl peroxide (BPO) and a retinoid, at least one of which and by some embodiments both are encapsulated by a silica-based shell. International Application No. PCT/IL2010/001092 (published as WO 2011/080741) describes methods and compositions utilizing microcapsules having a stabilized core with encapsulated active agents, such as benzoyl peroxide and/or all-trans retinoic acid (ATRA). Both these International Applications are hereby incorporated by reference in their entirety.

The co-pending U.S. patent application Ser. No. 13/537,646 (published as US 2013/0095185) describes compositions for topical application, where the compositions comprise microcapsules having a core that comprises benzoyl peroxide and a shell that comprises an inorganic polymer and microcapsules having a core that comprises a retinoid and a shell that comprises an inorganic polymer, and a stabilizing agent. The composition can be in a variety of forms, such as emulsion and gel. The benzoyl peroxide and/or all-trans retinoic acid (ATRA) stabilities in the compositions of the co-pending U.S. application Ser. No. 13/537,646 (published as US2013/0095185) are different and much improved over the compositions described in the International Application No. PCT/IL/2008/000140 (published as WO 2008/093346) and U.S. Pat. No. 8,617,580.

The compositions described in the co-pending U.S. application Ser. No. 13/537,646 (published as US2013/0095185) are in fact the first instance of a tretinoin/BPO composition exhibiting long term stability suitable for commercial use. For comparison, a 2010 publication (Del Rosso et al. J. Clin. Aesthet. Dermatol. 2010, October; 3(10): 26-28) (3 years after the priority date of WO 2008/093346 and U.S. Pat. No. 8,617,580) hails stability of the tretinoin in the tretinoin/BPO composition for 1-7 hours as a success. Of course, 1-7 hrs stability is not good enough for a commercial product. The composition used in Del Rosso et al. is actually a mixture of a 0.05% tretinoin gel (Atralin Gel, Coria Laboratories) with BPO gel. Atralin is formulated as an aqueous gel and BPO is not encapsulated. This may explain the short stability period of the Del Rosso et al. combination composition.

There is a long felt and unmet need for an ATRA/BPO composition suitable for commercial use, exhibiting long term stability.

### SUMMARY OF THE INVENTION

Various methods and compositions utilizing microcapsules having a stabilized core with encapsulated active agents are known. Although various embodiments of such compositions containing encapsulated BPO and encapsulated ATRA are useful for their intended purpose, during additional research and product development involving these and other microencapsulated BPO/retinoid compositions, it was discovered that some formulations exhibited certain shelf life problems, e.g., physical stability, retinoid stability and/or microbial content problems, particularly after long-term storage and/or elevated storage temperatures. The extent and source of these problems had not been previously recognized. For example, it was believed that compositions containing BPO would be unlikely to have microbial content problems because BPO is a strong oxidizing agent and effective antimicrobial. Initial attempts to address the microbial content problem by including an antimicrobial were complicated by the discovery that some antimicrobials resulted in retinoid degradation. Initial attempts to address the physical stability problem by including a surface active stabilizer in the composition were likewise complicated by the discovery that some stabilizers also resulted in retinoid degradation.

The inventors of the present invention have found that it is possible to provide compositions comprising both benzoyl peroxide (BPO) and all-trans retinoic acid (ATRA), each in separated encapsulated forms, having physical stability, chemical stability and/or microbial stability.

This invention provides methods of treatment of an acne patient in need thereof by the concomitant topical administration of BPO and ATRA. While the concomitant topical administration may be carried out in several alternative ways, the acne patient receives concomitant (same time) application of both actives BPO and ATRA as a mixture.

The concomitant topical administration may be carried out by applying to the acne skin lesions a single composition comprising both BPO and ATRA or from a dual chamber dispenser containing BPO in one chamber and ATRA in a second chamber or from two separate dispensers, one containing a BPO composition and the other an ATRA composition, mixed on the skin of the patient.

In its first aspects the present invention provides a composition for topical application, comprising: a plurality of first core-shell microcapsules comprising a first core that comprises benzoyl peroxide and a first shell that comprises a first inorganic polymer; and a plurality of second core-shell microcapsules comprising a second core that comprises a retinoid and a second shell that comprises a second inorganic polymer; wherein said composition is an oil in water emulsion comprising a polyoxylstearate and a glycerylstearate wherein the ratio of said polyoxylstearate to said glycerylstearate is in the range of 0.1:10 to 10:0.1. In an embodiment, the ratio of said polyoxylstearate to said glycerylstearate is in the range of 1:10 to 10:1. In another embodiment, the ratio of said polyoxylstearate to said glycerylstearate is in the range of 1:5 to 5:1. In another embodiment, the ratio of said polyoxylstearate to said glycerylstearate is in the range of 2:3 to 3:2.

In a further aspect the invention provides a composition for topical application, comprising: a plurality of first core-shell microcapsules comprising a first core that comprises benzoyl peroxide and a first shell that comprises a first inorganic polymer; and a plurality of second core-shell microcapsules comprising a second core that comprises a retinoid and a second shell that comprises a second inorganic polymer; wherein said composition is in a gel form comprising at least one non-ionic polymeric dispersant and at least one thickening agent.

In yet a further aspect the invention provides a composition for topical application, comprising: a plurality of first core-shell microcapsules comprising a first core that comprises benzoyl peroxide and a first shell that comprises a first inorganic polymer; a plurality of second core-shell microcapsules comprising a second core that comprises a retinoid and a second shell that comprises a second inorganic polymer; and an amount of a stabilizing agent that is effective to: (i) maintain the amount of benzoyl peroxide and the amount of retinoid in the composition at a level that is at least about 90% of the initial amounts, as measured after storage of the composition at a storage condition of 30° C. for three months; and (ii) maintain the microbial count of the composition at a level that meets the criteria of USP 51, as measured by Antimicrobial Effectiveness Testing (AET) after storage of the composition at a storage condition of 40° C. for one month.

In yet a further aspect the invention provides a composition in a gel form for topical application, comprising: a plurality of first core-shell microcapsules comprising a first core that comprises benzoyl peroxide and a first shell that comprises a first silica polymer; a plurality of second core-shell microcapsules comprising a second core that comprises all-trans retinoic acid (ATRA) and a second shell that comprises a second silica polymer; and at least one non-ionic polymer in an amount effective to provide at least one stabilization selected from the group consisting of (i) viscosity stabilization whereby the viscosity of said composition is maintained such that the change in viscosity of said composition, as measured after manufacture followed by three months storage at 25° C., is less than about 30%, and (ii) degradation stabilization whereby the amount of degradation of said ATRA, as measured after manufacture followed by three months storage at 25° C., is less than about 10%.

In another aspect the invention provides a composition in an emulsion form for topical application, comprising: a plurality of first core-shell microcapsules comprising a first core that comprises benzoyl peroxide and a first shell that comprises a first silica polymer; a plurality of second core-shell microcapsules comprising a second core that comprises all-trans retinoic acid (ATRA) and a second shell that comprises a second silica polymer; and a stabilizing agent comprising at least one non-ionic surfactant and at least one antimicrobial in amounts effective to: (i) maintain the amount of benzoyl peroxide and the amount of retinoid in the composition at a level that is at least about 90% of the initial amounts, as measured after storage of the composition at a storage condition selected from the group consisting of 40° C. for two months, 30° C. for three months, 25° C. for six months and 5° C. for two years; and (ii) maintain the microbial count of the composition at a level that meets the criteria of USP 51, as measured by Antimicrobial Effectiveness Testing (AET) after storage of the composition at a storage condition of 40° C. for one month.

The invention also provides a composition in an emulsion form for topical application, comprising: a plurality of first core-shell microcapsules comprising a first core that comprises benzoyl peroxide and a first shell that comprises a first silica polymer, the benzoyl peroxide being present in the composition in an initial amount of about 6% by weight, based on the total weight of the composition; a plurality of second core-shell microcapsules comprising a second core that comprises all-trans retinoic acid (ATRA) and a second shell that comprises a second silica polymer, the ATRA being present in the composition in an initial amount of about 0.1% by weight, based on the total weight of the composition; and a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea; wherein the stabilizing agent is present in an amount effective to: (ia) maintain the amount of benzoyl peroxide at a level of at least about 5.4% by weight based on the total weight of the composition, as measured after storage of the composition at a storage condition selected from the group consisting of 40° C. for two months, 30° C. for three months, 25° C. for six months and 5° C. for two years; (ib) maintain the amount of ATRA at a level of at least about 0.09% by weight based on the total weight of the composition, as measured after storage of the composition at a storage condition selected from the group consisting of 40° C. for two months, 30° C. for three months, 25° C. for six months and 5° C. for two years; (ii) maintain the microbial count of the composition at a level that meets the criteria of USP 51, as measured by Antimicrobial Effectiveness Testing (AET) after storage of the composition at a storage condition of 40° C. for one month; and (iii) maintain the bulk viscosity of said composition at about one million cps or less and within a range of about 70% to about 130% of the initial bulk viscosity, as measured after storage of the composition at 25° C. for three months.

According to another one of its aspects the invention provides a composition in an emulsion form for topical application, comprising: a plurality of first core-shell microcapsules comprising a first core that comprises benzoyl peroxide and a first shell that comprises a first silica polymer, the benzoyl peroxide being present in the composition in an initial amount of about 3% by weight, based on the total weight of the composition; a plurality of second core-shell microcapsules comprising a second core that comprises all-trans retinoic acid (ATRA) and a second shell that comprises a second silica polymer, the ATRA being present in the composition in an initial amount of about 0.05% by weight, based on the total weight of the composition; and a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea; wherein the stabilizing agent is present in an amount effective to: (ia) maintain the amount of benzoyl peroxide at a level of at least about 2.7% by weight based on the total weight of the composition, as measured after storage of the composition at a storage condition selected from the group consisting of 40° C. for two months, 30° C. for three months, 25° C. for six months and 5° C. for two years; (ib) maintain the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years; (ii) maintain the microbial count of the composition at a level that meets the criteria of USP 51, as measured by Antimicrobial Effectiveness Testing (AET) after storage of the composition at a storage condition of 40° C. for one month; and (iii) maintain the bulk viscosity of said composition at about one million cps or less and within a range of about 70% to about 130% of the initial bulk viscosity, as measured after storage of the composition at 25° C. for three months.

The invention further provides a packaged product, comprising a sealable container and a composition as described herein contained within the sealable container.

These aspects and other embodiments thereof are described in greater detail below.

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIG. 1 illustrates the ATRA and BPO contents of a series of formulations of the invention prepared according to Examples 15-33, after 4-week storage at 40 C. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown.

FIG. 2 illustrates the bulk viscosity of formulations of the invention prepared according to Examples 15-33, after 4-week storage at 40 C. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown.

FIG. 3 illustrates the ATRA and BPO contents of a series of formulations of the invention prepared according to Examples 15-33, after 4-week storage at 30 C. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown.

FIG. 4 illustrates the ATRA and BPO contents of a series of formulations of the invention prepared according to Examples 15-33, after 4-week storage at 25 C. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown.

FIG. 5 illustrates the bulk viscosity of formulations of the invention prepared according to Examples 15-33, after 4-week storage at 25 C. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown.

### DETAILED DESCRIPTION

The present invention provides methods and regimens of administration for acne treatment, comprising concomitant topical administration of benzoyl peroxide (BPO) and all trans retinoic acid (ATRA).

This invention provides methods of treatment of an acne patient in need thereof by the concomitant topical administration of BPO and ATRA.

While the concomitant topical administration may be carried out in several alternative ways, the acne patient always receives concomitant (same time) application of both actives BPO and ATRA as a mixture.

The concomitant topical administration may be carried out by either
1. Application to the acne skin lesions of a single composition comprising both BPO and ATRA or
2. Application to the skin of a patient of an instantly mixed composition from a dual chamber dispenser containing BPO in one chamber and ATRA in a second chamber, mixed before application to the skin of a patient or
3. Application to the acne skin lesions of two compositions from two separate dispensers, one containing a BPO composition and the other an ATRA composition, mixed on the skin of the patient.

Some of the formulations of the single compositions have been disclosed in the co-pending U. S. Patent application Ser. No. 13/537,646 (published as US2013/0095185).

The formulations and the administration of the two separate compositions of #2 and #3 above are described in Examples 35-38.

The compositions described in the co-pending U.S. application Ser. No. 13/537,646 (published as US2013/0095185) are in fact the first instance of a tretinoin/BPO composition exhibiting long term stability suitable for commercial use.

Without wishing to be bound by theory, we believe the long term stability of some the compositions of the present invention are due to the encapsulation of both actives ATRA and BPO and the improved formulation of this invention. This is in contrast to the short term stability of the Del Rosso compositions (Del Rosso et al. J. Clin. Aesthet. Dermatol. 2010, October; 3(10): 26-28) which exhibited stability for 1-7 days.

In an embodiment, there is provided a stable topical composition comprising BPO and ATRA, in which the initial concentration of BPO is 3% w/w and the initial concentration of ATRA is 0.05% w/w and wherein the composition maintains the amount of benzoyl peroxide at a level of at least about 2.7% by weight based on the total weight of the composition, as measured after storage of the composition at a storage condition selected from the group consisting of 40° C. for two months, 30° C. for three months, 25° C. for six months and 5° C. for two years and maintains the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years; (ii) maintain the microbial count of the composition at a level that meets the criteria of USP 51, as measured by Antimicrobial Effectiveness Testing (AET) after storage of the composition at a storage condition of 40° C. for one month; and maintains the bulk viscosity of said composition at about one million cps or less and within a range of about 70% to about 130% of the initial bulk viscosity, as measured after storage of the composition at 25° C. for three months.

We have conducted a stability study of topical ATRA/BPO compositions wherein, in contrast to the other stable compositions of the present disclosure, only one of the two actives (BPO) or none are encapsulated (see Example 34 of the present disclosure).

A composition comprising non-encapsulated BPO 2.5% and non-encapsulated tretinoin 0.1% was found to be stable for at least 2 months at 25° C./65% RH and 40° C./75% RH.

Without wishing to be bound by theory, we believe that the enhanced stability of the non-encapsulated compositions of this invention is due to the particle size of the two actives BPO and ATRA. Larger particle size of the two actives leads to more stable compositions and reduced irritation side-effects.

The results of this stability study demonstrated that when none of the two actives was encapsulated, but have larger particle size, the composition comprising the two actives was stable for at least two months, which is much better than the poor stability results (1-7 days) of the Del Rosso (2010) compositions.

Dispensing the ATRA/BPO Compositions from a Dual-Chamber Dispenser

The compositions consisting of a first composition comprising BPO and a second composition comprising ATRA, concomitantly administered once daily from a dual chamber dispenser, are disclosed in Examples 35-37.

The dual chamber dispenser may be disposable (for one use) or for multiple uses.

The dual chamber dispenser may be a commercially available device or a dispenser custom manufactured for the compositions of this invention, made of materials resistant to the two actives and the compositions of this invention and having chamber volumes fit for the methods of treatment and regimens of administration disclosed herein.

Such dual chamber dispensers have been disclosed, for example, in U.S. Pat. No. 6,117,433 or U.S. Patent Application No. 2004/0157766 (now abandoned).

In an embodiment, there is provided a dual chamber dispenser having a first chamber charged with a first composition comprising from about 0.01% w/w to about 0.1% w/w of solid particulate all trans retinoic acid (ATRA), and a second chamber charged with a second composition comprising from about 2% w/w to about 10% w/w of solid particulate benzoyl peroxide (BPO), wherein the two compositions are mixed before applying on the skin of a patient in need thereof.

Dispensing the ATRA and BPO Compositions, Instantly Mixed on the Skin of a Patient from Two Separate Dispensers, One Containing a BPO Composition and the Other an ATRA Composition

This concomitant administration of BPO and ATRA is carried out by application to the skin of a patient an instantly mixed composition from two separate dispensers (Example 38), one containing a BPO composition and the other an ATRA composition. The two compositions are instantly mixed on the skin of the patient.

The methods for acne treatment of this invention comprise concomitant topical administration of benzoyl peroxide (BPO) and all trans retinoic acid (ATRA) for up to 2 weeks, up to 1 month, preferably up to 2 months and more preferably up to 3 months.

The regimens of administration for acne treatment of this invention comprise daily concomitant application of the compositions of this invention on the skin of a patient in need thereof for a period of up to 2 weeks, up to 1 month, preferably up to 2 months and more preferably up to 3 months.

The compositions used for these regimens of administration may be either a single composition comprising both BPO and ATRA, or alternatively, a first composition comprising BPO and a second composition comprising ATRA, concomitantly administered once daily from a dual chamber dispenser and mixed before applying on the skin of a patient in need thereof, or alternatively by application to the skin of a patient of an instantly mixed composition from two separate dispensers, one containing a BPO composition and the other an ATRA composition. The two compositions are mixed on the skin of the patient.

Some embodiments provide a composition for topical application, where the composition contains: microcapsules having a core that comprises BPO and a shell that comprises an inorganic polymer, microcapsules having a core that comprises a retinoid and a shell that comprises an inorganic polymer, and a stabilizing agent. The stabilizing agent provides physical stabilization (e.g., bulk viscosity stabilization), chemical stabilization (e.g., active ingredient stabilization) and/or antimicrobial stabilization to the composition, as described in greater detail below. The composition can be in a variety of forms, including, but not limited to, an emulsion form, a cream form, an aqueous solution form, an oil form, an ointment form, a paste form, a gel form, a lotion form, and a suspension form. In some embodiments, the microcapsules having a core that comprises a retinoid or BPO can be in the form of an emulsion prior to formation of the composition. In further embodiments, these emulsions may be incorporated into a cream, gel, lotion, or other form providing the composition described above.

In some embodiments, the composition is in a gel form. It has been surprisingly discovered that a gel formulation comprising both benzoyl peroxide and retinoid in separated encapsulated forms was found to provide retinoid stability in the presence of a non-ionic polymeric dispersant and a thickening agent in the gel formulation. For example, an embodiment provides a composition for topical application, comprising: a plurality of first core-shell microcapsules comprising a first core that comprises benzoyl peroxide and a first shell that comprises a first inorganic polymer; a plurality of second core-shell microcapsules comprising a second core that comprises a retinoid and a second shell that comprises a second inorganic polymer; wherein said composition is in a gel form comprising at least one non-ionic polymeric dispersant and at least one thickening agent. As illustrated in the Examples below, in some embodiments the stability of the retinoid in a gel that contains a non-ionic polymeric dispersant (such as PVP) is surprisingly superior to that of the retinoid in the gel form that does not contain a non-ionic polymeric dispersant.

In other embodiments, the composition is in an emulsion form. It has been surprisingly found that in some embodiments, the retinoid in the emulsion is surprisingly stable. This finding is contrary to the expectations of the skilled artisan who would have instead expected the oil and surfactants in the emulsion to increase the rate of leaching of the retinoid out of its protective microcapsule. Surprisingly, no such increase in leaching was observed. For example, an embodiment provides a composition for topical application, comprising: a plurality of first core-shell microcapsules comprising a first core that comprises benzoyl peroxide and a first shell that comprises a first inorganic polymer; and a plurality of second core-shell microcapsules comprising a second core that comprises a retinoid and a second shell that comprises a second inorganic polymer; wherein said composition is an oil in water emulsion comprising a polyoxylstearate and a glycerylstearate, wherein the ratio of said polyoxylstearate to said glycerylstearate is in the range of 0.1:10 to 10:0.1. In an embodiment, the ratio of said polyoxylstearate to said glycerylstearate is in the range of 1:10 to 10:1. In another embodiment, the ratio of said polyoxylstearate to said glycerylstearate is in the range of 1:5 to 5:1. In another embodiment, the ratio of said polyoxylstearate to said glycerylstearate is in the range of 2:3 to 3:2. As illustrated in the Examples below, in some embodiments the stability of the retinoid in the emulsion form is surprisingly comparable to that of the retinoid in the gel form.

Various natural oils and synthetic oils (such as silicone oils) and mixtures thereof can be used as the oil component of the oil in water emulsion. Non-limiting examples of synthetic oils include paraffin oil, isopropyl myristate, caprylic/capric triglyceride, silicone oil (such as dimethicone and cyclomethicone) and mixtures thereof. Non-limiting examples of natural oils include squalane, almond oil, castor oil, olive oil, jojoba oil, sunflower oil, soybean oil, grape seed oil and mixtures thereof. Amounts of oil in the composition can be in the range of about 0.05% w/w to about 50% w/w. In an embodiment, amounts of oil in the composition can be in the range of about 0.5% w/w to about 20% w/w. In an embodiment, amounts of oil in the composition can be in the range of about 1% w/w to about 10% w/w.

Gel and emulsion forms of the composition can optionally contain other ingredients. For example, in an embodiment, the composition can contain at least one humectant. Non-limiting examples of humectants include water soluble humectants selected from the group consisting of propylene glycol, glycerin, polyethylene glycol-X, and mixtures thereof, where X indicates the average number of ethylene glycol units and is in the range of 200 to 10,000.

### Microcapsules

As used herein, the term “microcapsule” refers to any micro- or nano-sized particle having a core-shell structure that is capable of encasing, encapsulating or entrapping compounds, including but not limited to active ingredients such as BPO and/or a retinoid (e.g., ATRA). In some embodiments, microcapsules are made by a sol-gel process, e.g., as generally described in WO 03/034979 and WO 2011/080741.

### Core

As used herein, the term “core” refers to the inside part of a microcapsule comprising at least one active ingredient surrounded by a shell of the microcapsule. In some embodiments, the core can be solid at room temperature. In other embodiments, the core can be in a semi-solid phase at room temperature. In some embodiments, the core can be in the form of an emulsion, for example an oil-in-water emulsion. In some embodiments, the core can be in the form of oil solution. In some embodiments, the core can be in the form of an aqueous solution. In some embodiments, the core can be in the form of a dispersion.

Additional compound(s) can be present in the core. Non-limiting examples of the additional compounds that can be present in the core include phase changing materials (PCMs), carriers, excipients, antioxidants, pharmaceutically acceptable polymers, and salts. In some embodiments, the core comprises at least one phase changing material. As described elsewhere herein in greater detail, exemplary phase changing materials include, but are not limited to, natural and synthetic paraffins; C₁₀-C₁₀₀ (straight, branched, and cyclic) alkanes, alkenes and alkynes; C₁₀-C₁₀₀ aliphatic alcohols (e.g., fatty alcohols); fatty acids; carnauba wax; beeswax; and mixtures thereof. In some embodiments, the core comprises at least one antioxidant. Examples of antioxidants include, but are not limited to, butylated hydroxytoluene (BHT), butylated hydroxyanisole (BHA), vitamin E, vitamin E acetate, vitamin E palmitate, vitamin C, an ester of vitamin C, and one or more salts of vitamin C.

### Shell

As used herein, the term “shell” refers to the part of a microcapsule that surrounds the core of the microcapsule. In some embodiments, the shell comprises an inorganic polymer (for example, a silica polymer). In some embodiments, the inorganic polymer can be prepared from a sol-gel precursor.

As used herein, the term “sol-gel precursor” refers to any metal or semi-metal organo-metallic monomer, or a prepolymer (which means several monomers polymerized together) thereof, which provide a glass or ceramic material by in-situ polymerization (an inorganic sol-gel polymerization process). In some embodiments, the sol-gel precursor can be a metal or semi-metal organo-metallic monomer. Examples of sol-gel precursor include, but are not limited to, a metal alkoxide monomer; a semi-metal alkoxide monomer; a metal ester monomer; a semi-metal ester monomer; a silazane monomer; a colloidal silica; a monomer of the formula M(R)ₙ(P)ₘ, where M can be a metallic or a semi-metallic element, R can be a hydrolyzable substituent, n can be an integer from 2 to 6, P can be a non polymerizable substituent, and m can be an integer from 0 to 6; and a partially hydrolyzed and partially condensed polymer thereof. Various metallic or semi metallic elements can be used in the sol-gel precursor, for example, Si, Ti, Zr, Al, and Zn. Examples of semi-metal alkoxide monomers include, but are not limited to, tetramethoxysilane (also known as tetramethyl orthosilicate or TMOS), tetraethoxysilane (also known as tetraethyl orthosilicate or TEOS), dimethyldimethoxysilane, methyltrimethoxysilane, diethyldimethoxysilane, and sodium silicate.

In some embodiments, the sol-gel precursor can be selected from a silicon alkoxide monomer; a silicon ester monomer; a monomer of the formula Si(R)ₙ(P)ₘ, wherein R can be a hydrolyzable substituent, n can be an integer from 2 to 4, P can be a non polymerizable substituent, and m can be an integer from 0 to 4; a partially hydrolyzed and partially condensed polymer of any of the above, and mixtures of any of the above. Non-limiting examples of silicon alkoxide monomer include tetramethoxy silane, tetraethoxy silane, and combinations thereof. Non-limiting examples of monomers of the formula Si(R)ₙ(P)ₘ include methyl trimethoxysilane, dimethyl dimethoxysilane, and combinations thereof.

### Active Ingredient

As used herein, the term “active ingredient” or “active” or “active agent” refers to a molecule or substance that can be used in medicine and/or cosmetics and which provides to the final product at least one desired property. Amounts of active ingredients in the compositions described herein are expressed in terms of weight percentage of the active ingredients in the composition based on the total weight of the composition, unless otherwise stated. Examples of active ingredients include but are not limited to BPO and retinoids (e.g., ATRA). The active ingredient can be present in the composition described herein in a variety of concentrations. For example, in certain embodiments, the amount of active ingredient in the composition can be about 0.001%, about 0.005%, about 0.01%, about 0.05%, about 0.1%, about 0.5%, about 1%, about 3%, about 5%, about 6%, about 7%, about 8%, about 9%, about 10%, about 15%, about 20%, or ranges between any two of these values, by weight, based on total weight of the composition. In some embodiments, the amount of active ingredient can be in the range of about 0.01% to about 1% by weight, based on total weight of the composition. In some embodiments, the amount of active ingredient can be in the range of about 1% to about 10% by weight, based on total weight of the composition. In particular embodiments, the amount of active ingredient can be about 3% by weight or about 6% by weight, based on total weight of the composition. In other embodiments, the amount of active ingredient can be about 0.1% by weight or about 0.05% by weight, based on total weight of the composition. In embodiments where multiple active ingredients are present, any combination of the foregoing amounts can be included.

The active ingredient can be a pharmaceutical agent, a cosmetic agent, a dermatological agent, an agrochemical agent, or any combination of the foregoing. In particular embodiments, the pharmaceutical agent, cosmetic agent, and/or dermatological agent can be an anti-acne agent. Examples of active ingredients include, but are not limited to, benzoyl peroxide (BPO) and a retinoid. Examples of retinoids include, but are not limited to, all-trans-retinol, all-trans-retinoic acid (also known as tretinoin or ATRA), retinol, retinal, isotretinoin, alitretinoin, etretinate (and its metabolite acitretin), tazarotene, bexarotene, and adapalene. In some embodiments, the active ingredient can be benzoyl peroxide. In some embodiments, the active ingredient can be a retinoid. In some embodiments, the active ingredient can be ATRA. In some embodiments, ATRA can be present in the composition in an amount in the range of about 0.01% to about 0.1% by weight, based on total weight of the composition. In particular embodiments, the retinoid can be present in the composition in an amount in the range of about 0.05% by weight or about 0.1% by weight, based on the total weight of the composition. In some embodiments, benzoyl peroxide can be in the composition in an amount in the range of about 1% to about 10%/c by weight, based on total weight of the composition. In particular embodiments, the BPO can be present in the composition in an amount of about 6% by weight, based on the total weight of the composition.

In other embodiments, the BPO can be present in the composition in an amount of about 3% by weight, based on the total weight of the composition.

In an embodiment, the amount of BPO can be about 6% by weight and the amount of retinoid can be about 0.1% by weight. An exemplary embodiment can be a composition in an emulsion form for topical application, comprising:

a plurality of first core-shell microcapsules comprising a first core that comprises benzoyl peroxide and a first shell that comprises a first silica polymer, the benzoyl peroxide being present in the composition in an initial amount of about 6% by weight, based on the total weight of the composition;

a plurality of second core-shell microcapsules comprising a second core that comprises all-trans retinoic acid (ATRA) and a second shell that comprises a second silica polymer, the ATRA being present in the composition in an initial amount of about 0.1% by weight, based on the total weight of the composition; and

a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea;

wherein the stabilizing agent is present in an amount effective to:

(ia) maintain the amount of benzoyl peroxide at a level of at least about 5.4% by weight based on the total weight of the composition, as measured after storage of the composition at a storage condition selected from the group consisting of 40° C. for two months, 30° C. for three months, 25° C. for six months and 5° C. for two years;

(ib) maintain the amount of ATRA at a level of at least about 0.09% by weight based on the total weight of the composition, as measured after storage of the composition at a storage condition selected from the group consisting of 40° C. for two months, 30° C. for three months, 25° C. for six months and 5° C. for two years;

(ii) maintain the microbial count of the composition at a level that meets the criteria of USP 51, as measured by Antimicrobial Effectiveness Testing (AET) after storage of the composition at a storage condition of 40° C. for one month; and

(iii) maintain the bulk viscosity of said composition at about one million cps or less and within a range of about 70% to about 130% of the initial bulk viscosity, as measured after storage of the composition at 25° C. for three months.

In another embodiment, the amount of BPO can be about 3% by weight and the amount of retinoid can be about 0.05% by weight. An exemplary embodiment can be a composition in an emulsion form for topical application, comprising:

a plurality of first core-shell microcapsules comprising a first core that comprises benzoyl peroxide and a first shell that comprises a first silica polymer, the benzoyl peroxide being present in the composition in an initial amount of about 3% by weight, based on the total weight of the composition;

a plurality of second core-shell microcapsules comprising a second core that comprises all-trans retinoic acid (ATRA) and a second shell that comprises a second silica polymer, the ATRA being present in the composition in an initial amount of about 0.05% by weight, based on the total weight of the composition; and

a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea;

wherein the stabilizing agent is present in an amount effective to:

(ia) maintain the amount of benzoyl peroxide at a level of at least about 2.7% by weight based on the total weight of the composition, as measured after storage of the composition at a storage condition selected from the group consisting of 40° C. for two months, 30° C. for three months, 25° C. for six months and 5° C. for two years;

(ib) maintain the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years;

(ii) maintain the microbial count of the composition at a level that meets the criteria of USP 51, as measured by Antimicrobial Effectiveness Testing (AET) after storage of the composition at a storage condition of 40° C. for one month; and

(iii) maintain the bulk viscosity of said composition at about one million cps or less and within a range of about 70% to about 130% of the initial bulk viscosity, as measured after storage of the composition at 25° C. for three months.

In some embodiments, the microcapsule can be configured to release at least one active ingredient encapsulated in the core by extraction. As used herein, the term “extraction” refers to an action or mechanism that induces the active ingredient to be released from its encapsulant upon topical application. In some embodiments, the extraction can be induced by a rubbing or spreading action.

In some embodiments, the extraction can be induced via drying of the composition. In some embodiments, the extraction can be induced by contacting the microcapsules with a fat, a lipid, and/or an oil. In some embodiments, the fat, lipid, and/or oil are present in the skin. In some embodiments, the extraction can be induced by contacting the microcapsule with water, an electrolyte, a surfactant, a buffering agent, or any mixture thereof. In some embodiments, the water and electrolyte are present in a bodily fluid, such as sweat, or are present on the surface of the skin. In some embodiments, the electrolyte, the surfactant, the buffering agent, or the mixtures thereof are added to the composition prior to the topical application.

### Stabilizing Agent

It has now been discovered that various compounds, referred to herein as a “stabilizing agent(s)”, when used in an effective amount in a composition as described herein, can provide various types of stabilization, including active ingredient stabilization, bulk viscosity stabilization and/or antimicrobial stabilization. For example, in various embodiments an effective amount of a stabilizing agent in a composition as described herein can at least one of the following types or degrees of stabilization:

(a) maintain the amount of active ingredients (e.g., amount of BPO and the amount of retinoid) in the composition at a level that is at least about 90% of the initial amounts, as measured after storage of the composition at a storage condition selected from the group consisting of 40° C. for two months, 30° C. for three months, 25° C. for six months and 5° C. for two years;

(b) maintain the amount of the amount of active ingredients (e.g., amount of BPO and the amount of retinoid in the composition at a level that is at least about 97% of the initial amounts, as measured after storage of the composition at a storage condition of 5° C. for two years;

(c) maintain the microbial count of the composition at a level that meets the criteria of USP 51, as measured by Antimicrobial Effectiveness Testing (AET) after storage of the composition at a storage condition of 40° C. for one month;

(d) maintain the microbial count of the composition at a level that meets the criteria of USP 61, as measured by Microbial Limits Testing (MLT) after storage of the composition at a storage condition of 25° C. for six months;

(e) maintain the bulk viscosity of the composition at a level selected from the group consisting of about one million cps or less, about 500,000 or less, and about 300,000 or less, as measured after storage of the composition at 25° C. for three months;

(f) maintain the bulk viscosity of the composition at a level selected from the group consisting of about 60,000 cps or more, about 80,000 or more, and about 120,000 or more, as measured after storage of the composition at 25° C. for three months;

(g) maintain the bulk viscosity of the composition at a level within a range of about 70% to about 130% of the initial bulk viscosity, as measured after storage of the composition at 25° C. for three months.

A stabilizer can be a single compound or a mixture of compounds. In some embodiments a stabilizer can provide a single type of stability, such as active ingredient stabilization, bulk viscosity stabilization or antimicrobial stabilization, including but not limited to one of stabilization types (a) through (g) above. In other embodiments a stabilizer can provide two or more types of stability, such as two or more of active ingredient stabilization, bulk viscosity stabilization or antimicrobial stabilization, including but not limited to a plurality of stabilization types (a) through (g) above. In some embodiments an effective amount of a stabilizer provides a particular type or types of stability, and a different amount provides a different type or types of stability. Routine experimentation guided by the teachings provided herein can be used to identify compounds and mixtures thereof that provide one or more types of stability, and thus all combinations of effective stabilizing agents and types of stabilization are encompassed by the descriptions provided herein.

Various compounds can be used as a stabilizing agent, including non-ionic surfactants, water-soluble non-ionic polymers (including non-ionic polymeric dispersants and water-soluble non-ionic polymers), antimicrobials, thickening agents (including ionic and non-ionic thickening agents) and mixtures thereof.

Non-limiting examples of non-ionic surfactants useful as stabilizing agents include but are not limited to polyoxylstearates and glycerylstearates. Non-limiting examples of suitable polyoxylstearates include polyoxyl-8 stearate, polyoxyl-20 stearate, polyoxyl-40 stearate, polyoxyl-100 stearate, and mixtures thereof. Non-limiting examples of suitable glycerylstearates include glyceryl mono-stearate, glyceryl di-stearate, and mixtures thereof. Amounts of polyoxylstearates and/or glycerylstearates in the composition (such as in an emulsion composition), can be in the range of about 0.1% w/w to about 30% w/w. In an embodiment, amounts of polyoxylstearates and/or glycerylstearates in the composition can be in the range of about 0.5% w/w to about 10% w/w. In an embodiment, amounts of polyoxylstearates and/or glycerylstearates in the composition can be in the range of about 1% w/w to about 5% w/w.

Non-limiting examples of water-soluble non-ionic polymers include non-ionic polymeric dispersants such as (but not limited to) polyvinyl pyrrolidone (PVP), polyvinyl pyrrolidone-co-vinyl acetate, polyamide, polyurethane, polyurea, and mixtures thereof. Amounts of water-soluble non-ionic polymers (such as polymeric dispersants) in the composition can be in the range of about 0.05% w/w to about 20% w/w. In an embodiment, amounts of water-soluble non-ionic polymers in the composition can be in the range of about 0.1% w/w to about 10% w/w. In an embodiment, amounts of water-soluble non-ionic polymers in the composition can be in the range of about 0.5% w/w to about 5% w/w.

Non-limiting examples of thickening agents include hydroxy propyl cellulose (HPC), hydroxyl ethyl cellulose (HEC), hydroxyl methyl cellulose (HMC), polyacrylic acid homopolymer, polyacrylic acid copolymer, silica and its derivatives, xanthan gum, arabic gum, polyvinyl alcohol, fatty alcohols, veegum, laponite, clay, and mixtures thereof. Polyacrylic acid polymers and copolymers may be referred to herein as carbomers, which are commercially available under various tradenames such as CARBOMER 934, CARBOMER 941, CARBOMER 934P, CARBOPOL 910, CARBOPOL 934, CARBOPOL 941, etc. Amounts of thickening agents in the composition can be in the range of about 0.01% w/w to about 10% w/w. In an embodiment, amounts of thickening agents in the composition can be in the range of about 0.05% w/w to about 5% w/w. In an embodiment, amounts of thickening agents in the composition can be in the range of about 0.1% w/w to about 2% w/w. In an embodiment of a composition that contains both a polymeric dispersant and a thickening agent, the polymeric dispersant can be different from the thickening agent.

In an embodiment, the stabilizing agent is polyvinylpyrrolidone (PVP), polyoxyl 100 stearate, glycerol monostearate, methylparaben, propylparaben, phenoxyethanol, imidazolidinyl urea, or a mixture thereof. Other compounds useful as stabilizing agents can be identified by routine experimentation guided by the teachings provided herein. With respect to compositions that contain BPO as an active ingredient, the BPO is not considered to be a stabilizing agent even if it exhibits antimicrobial properties. Thus, reference herein to a stabilizing agent or antimicrobial in a composition that contains BPO as an active ingredient will be understood by those skilled in the art as a reference to a second antimicrobial that is not BPO.

The stabilizing agent can be a single compound or a mixture of compounds and can provide more than one type of stability, e.g., active ingredient stabilization, bulk viscosity stabilization and/or antimicrobial stabilization. For example, in an embodiment, the stabilizing agent can be polyoxyl 100 stearate, glycerol monostearate, or a mixture thereof, in an amount that provides active ingredient stabilization and bulk viscosity stabilization as described herein. In another embodiment, the stabilizing agent can be an antimicrobial, including but not limited to methylparaben, propylparaben, phenoxyethanol, imidazolidinyl urea, or a mixture thereof, in an amount that provides active ingredient stabilization and antimicrobial stabilization as described herein. In another embodiment, the stabilizing agent can include polyoxyl 100 stearate, glycerol monostearate, or a mixture thereof, and further include methylparaben, propylparaben, phenoxyethanol, imidazolidinyl urea, or a mixture thereof, in an amount that provides active ingredient stabilization, bulk viscosity stabilization and antimicrobial stabilization as described herein.

In some embodiments, the stabilizing agent can be present in an amount effective to maintain the viscosity of the composition. In some embodiments, the viscosity of the composition can be maintained such that the change in viscosity of the composition, as measured after manufacture followed by three months storage at about 25° C. can be less than about 300%. In some embodiments, the viscosity of the composition can be maintained such that the change in viscosity of the composition, as measured after manufacture followed by three months storage at about 25° C. can be less than about 20%. In some embodiments, the viscosity of the composition can be maintained such that the change in viscosity of the composition, as measured after manufacture followed by three months storage at about 5° C., can be less than about 30%.

In some embodiments, the stabilizing agent can be present in an amount effective to inhibit the degradation of at least one active ingredient in the composition. In some embodiments, the active ingredient can be a retinoid. In some embodiments, the active ingredient can be all-trans retinoic acid (ATRA). In some embodiments, the amount of degradation of ATRA, as measured after manufacture followed by three months storage at about 25° C. can be less than about 10%. In some embodiments, the amount of degradation of ATRA, as measured after manufacture followed by three months storage at about 25° C. can be less than about 5%. In some embodiments, the amount of degradation of ATRA, as measured after manufacture followed by three months storage at about 5° C. can be less than about 10%. In some embodiments, the amount of degradation of ATRA, as measured after manufacture followed by three months storage at about 5° C. can be less than about 5%.

Those skilled in the art appreciate that the value obtained for a bulk viscosity measurement depends on the instrument of measurement, spindle used, speed of the instrument, and temperature of measurement. The bulk viscosity measurements referred to herein are measured using a Brookfield LVDV-11+Pro viscometer equipped with a small sample adaptor, spindle #63 (LV3) or spindle #SC4-25 at 1 rpm and at temperature of 30° C.±0.5° C. Antimicrobial stability can be measured in accordance with the criteria of USP 51, as measured by Antimicrobial Effectiveness Testing (AET) after storage of the composition at a storage condition of 40° C. for one month, and/or antimicrobial stability can be measured in accordance with the criteria of USP 61, as measured by Microbial Limits Testing (MLT) after storage of the composition at a storage condition of 25° C. for six months.

### Phase Changing Material

As used herein, the term “phase changing material” (PCM) refers to any substance capable of changing its state of matter (phase), or at least its viscosity, in accordance with the temperature it is exposed to. PCMs typically have a high heat of fusion which enables them to melt and solidify at certain temperatures, and are capable of storing and releasing large amounts of energy. Heat is absorbed or released when the PCM material changes from solid to liquid and vice versa. When a PCM reaches the temperature at which it changes phase or viscosity (for example its melting temperature), it absorbs large amounts of heat but is maintained at almost constant temperature. The PCM continues to absorb heat without a significant rise in temperature until all the material is transformed to the liquid phase. When the ambient temperature around the resulting liquid material falls, the PCM solidifies, releasing its stored latent heat.

As described herein, a PCM is typically non-reactive with the active ingredient encapsulated in the microcapsule, the emulsion formed, and the shell of the microcapsule described herein. In some embodiments, the PCM can be an organic material. Examples of PCMs include, but are not limited to, natural or synthetic paraffins (typically compounds having a molecular formula of CₙH₂ₙ₊₂, (n=10-100)), C₁₀-C₁₀₀ straight, branched, and cyclic alkanes, C₁₀-C₁₀₀ straight, branched, and cyclic alkenes (compounds having the noted number of carbons and at least one double bond), straight and branched C₁₀-C₁₀₀ alkynes (compounds having the noted number of carbons and at least one triple bond), straight and branched aliphatic alcohols (typically compounds having a molecular formula of CH₃(CH₂)ₙOH or branched versions thereof, wherein n=10-100) and fatty acids (typically compounds having a molecular formula of CH₃(CH₂)₂ₙCOOH and branched versions thereof, wherein n=10-100), or any combinations thereof.

In some embodiments, the PCM can be a natural or a synthetic paraffin. In some embodiments, the PCM can be a C₁₀-C₁₀₀ aliphatic alcohol (for example, C₁₀, C₂₀, C₃₀, C₄₀, C₅₀, C₆₀, C₇₀, C₈₀, C₉₀ to C₁₀ aliphatic alcohol). In other embodiments, the PCM can be a C₁₀-C₁₀₀ aliphatic fatty acid (for example, C₁₀, C₂₀, C₃₀, C₄₀, C₅₀, C₆₀, C₇₀, C₈₀, C₉₀ to C₁₀₀ aliphatic fatty acids).

In an embodiment, the PCM can be at least one fatty alcohol. Non-limiting examples of fatty alcohols include octyl alcohol, 2-ethyl hexanol, nonyl alcohol, decyl alcohol, undecanol, dodecyl alcohol, tridecyl alcohol, tetradecyl alcohol, pentadecyl alcohol, cetyl alcohol, palmitoleyl alcohol, heptadecyl alcohol, cetostearyl alcohol, stearyl alcohol, isostearyl alcohol, elaidyl alcohol, oleyl alcohol, linoleyl alcohol, elaidolinolenyl alcohol, ricinoleyl alcohol, nonadecyl alcohol, arachidyl alcohol, heneicosyl alcohol, behenyl alcohol, erucyl alcohol, lignoceryl alcohol, ceryl alcohol, montanyl alcohol, cluytyl alcohol, myricyl alcohol, melissyl alcohol, geddyl alcohol, cetearyl alcohol and mixtures thereof. Amounts of fatty alcohol in the composition can be in the range of about 0.2%/o w/w to about 50% w/w. In an embodiment, amounts of fatty alcohol in the composition can be in the range of about 1% w/w to about 20% w/w. In an embodiment, amounts of fatty alcohol in the composition can be in the range of about 3% w/w to about 10% w/w.

Exemplary PCMs include, but are not limited to: Carnauba wax (m.p. 82-86° C.), Beeswax pure (m.p. 61-65° C.), Beeswax white pure, (m.p. 61-65° C.), Beeswax bleached technical (m.p. 61-65° C.), Montan wax bleached (m.p. 80-86° C.), Montan wax bleached, partially saponified (m.p. 99-105° C.), Montanic acid (m.p. 81-87° C.), Hydrocarbon wax synthetic (m.p. 106-114° C.), Microcrystalline wax (m.p. 89-95° C.), Microcrystalline wax (m.p. 76-82° C.), Hardwax partially saponified (m.p. 104-109° C.), Beeswax yellow (m.p. 61-66° C.), Polishing Wax (m.p. 78-84° C.), Castor wax (m.p. 83-89° C.), Microwax (m.p. 89-95° C.), Microwax (m.p. 80-86° C.), Microwax (m.p. 76-82° C.), Ozokerite (m.p. 72-79° C.), Microcrystalline wax, plastic (m.p. 76-82° C.), Microcrystalline wax, soft (m.p. 74-80° C.), Wax blend (m.p. 62-68° C.), Polyolefin wax (m.p. 65-75° C.), Lanolin, Shellac, Bayberry wax (m.p. 45° C.), Candelilla wax (m.p. 67-79° C.), Ouricury wax, Rice bran wax (m.p. 77-86° C.), Soy candle (wax), Paraffin (m.p. 47-64° C.), Chinese wax, and any combinations thereof.

In some embodiments, the core comprises at least one PCM. In some embodiments, the PCM can be a natural paraffin, a synthetic paraffin, an aliphatic alcohol, a fatty acid, an ester of an aliphatic alcohol, an ester of a fatty acid, or combinations thereof. In some embodiments, the ester of a fatty acid comprises natural or synthetic beeswax.

In some embodiments, the PCM can be in the core of obtained microcapsules and not incorporated in any part of the shell of the microcapsules described herein.

### Compositions for Topical Application

As used herein, the term “topical” application refers to an application onto the skin, hair, ears, and/or mucous membranes.

Some embodiments disclosed herein provide a composition for topical application, wherein the composition comprises: a plurality of first microcapsules having a core that comprises benzoyl peroxide and a shell that comprises an inorganic polymer, a plurality of second microcapsules having a core that comprises a retinoid and a shell that comprises an inorganic polymer, and a stabilizing agent.

Some embodiments disclosed herein provide a composition in a gel form for topical application, where the composition comprises: a plurality of first microcapsules having a core that comprises benzoyl peroxide and a shell that comprises a first silica polymer; a plurality of second microcapsules having a core that comprises all-trans retinoic acid (ATRA) and a shell that comprises a second silica polymer; and at least one non-ionic polymer in an amount effective to provide viscosity stabilization and/or degradation stabilization. In some embodiments, the viscosity stabilization means that the viscosity of the composition can be maintained such that the change in viscosity of the composition, as measured after manufacture followed by three months storage at about 25° C. can be less than about 30%/. In some embodiments, the degradation stabilization means that the amount of degradation of the ATRA, as measured after manufacture followed by three months storage at about 25° C. can be less than about 10%.

In some embodiments, the non-ionic polymer can be present in an amount effective to provide viscosity stabilization. In some embodiments, the viscosity stabilization can be effective to maintain the viscosity of the composition at more than about 20,000 cps, about 25,000 cps, about 30,000 cps, about 35,000 cps, about 40,000 cps, about 45,000 cps, or about 50,000 cps as measured after manufacture followed by 3 months storage at a storage temperature. The storage temperature can be about 5° C. or about 25° C. In some embodiments, the non-ionic polymer can be present in an amount effective to provide degradation stabilization. In some embodiments, the non-ionic polymer can be present in an amount such that the amount of degradation of ATRA can be less than about 10%, 8%, or 5%. In some embodiments, the non-ionic polymer can be polyvinylpyrrolidnone.

In certain embodiments, the non-ionic polymer can be present in an amount effective to provide the viscosity stabilization and degradation stabilization. Other non-ionic polymers and effective amounts thereof that provide the viscosity stabilization and/or the degradation stabilization may be identified by those skilled in the art using routine experimentation guided by the teachings provided herein. Non-limiting examples of suitable non-ionic polymers include polyvinyl pyrrolidone (PVP), polyvinyl pyrrolidone-co-vinyl acetate, polyamide, polyurethane, polyurea, and mixtures thereof.

Some embodiments provide a composition in an emulsion form for topical application, where the composition comprises: a plurality of first microcapsules having a core that comprises benzoyl peroxide and a shell that comprises a silica polymer; a plurality of second microcapsules having a core that comprises all-trans retinoic acid (ATRA) and a shell that comprises a silica polymer; and at least one non-ionic surfactant in an amount effective to provide viscosity stabilization and/or degradation stabilization.

In some embodiments, the non-ionic surfactant can be present in an amount effective to provide viscosity stabilization. In some embodiments, the viscosity stabilization can be effective to maintain the viscosity of the composition at more than about 60,000 cps, 70,000 cps, 80,000 cps, 90,000 cps, 100,000 cps, 110,000 cps, 120,000 cps, 130,000 cps, 140,000 cps, 150,000 cps, 160,000 cps, or 170,000 cps as measured after manufacture followed by three months storage at a storage temperature. The storage temperature can be about 5° C. or about 25° C. In some embodiments, the non-ionic surfactant can be present in an amount effective to provide degradation stabilization. In some embodiments, the non-ionic surfactant can be present in an amount such that the amount of degradation of ATRA can be less than about 10%, 8%, or 5%.

In some embodiments, the non-ionic surfactant can be present in an amount effective to provide the viscosity stabilization and the degradation stabilization. Non-limiting examples of non-ionic surfactants include polyoxylstearates and glycerylstearates. Non-limiting examples of polyoxylstearates include Polyoxyl-8 stearate, Polyoxyl-20 stearate, Polyoxyl-40 stearate, and Polyoxyl-100 stearate. Non-limiting examples of glycerylstearates include glyceryl mono-stearate, glyceryl di-stearate, and mixtures thereof. Other non-ionic surfactants and effective amounts thereof that provide the viscosity stabilization and/or the degradation stabilization may be identified by those skilled in the art using routine experimentation guided by the teachings provided herein. In some embodiments, the non-ionic surfactant comprises a combination of polyoxyl 100 stearate and glycerol monostearate.

Various stabilizations described herein, for example, the viscosity stabilization and/or the degradation stabilization, may be effective for a storage time beyond 3 months at a storage temperature. For example, in some embodiments, the various viscosity stabilizations and/or the degradation stabilizations described herein are effective for a storage time of about 6 months, about 9 months, about 12 months, about 15 months, about 18 months, or about 24 months. In some embodiments, the storage temperature can be about 5° C., or about 25° C.

In another aspect, the present disclosure provides methods of preparing a composition comprising microcapsules disclosed herein. Those skilled in the art will appreciate the manner in which the working examples set forth below provide a specific description of how to make particular compositions and components thereof. Those skilled in the art will also appreciate the manner in which the specific working examples can be generalized and adapted to produce the other compositions described herein and components thereof.

In yet another aspect, the present disclosure provides a method for treating a surface condition (e.g., a skin disease or disorder) in a subject in need thereof, comprising topically administering to the subject an effective amount of a composition as described herein. Non-limiting examples of surface conditions that can be treated by topical application of effective amounts of the compositions described herein include acne, rosecea, psoriasis, photoaged skin, hyperpigmented skin, mucosal infected areas, inflamed dermatitis, and combinations thereof. In this context, terms such as “treat,” “treating,” “treatment,” etc. include inhibiting the surface condition (e.g., by arresting its development), relieving the surface condition (e.g., causing regression) and/or relieving one or more conditions caused by the surface condition (e.g., reducing one or more symptoms). Effective amounts of the compositions described herein for treating various surface conditions can be determined by those skilled in the art in the usual manner, e.g., by clinical trials, with appropriate adjustments by skilled clinicians in individual cases.

In yet another aspect, the present disclosure provides a packaged product, comprising a sealable container and a composition as described herein that is contained within the sealable container. The sealable container can have many different configurations, e.g., including but not limited to the various types of containers that are used for packaging cream, gel and ointment products for consumer use. Non-limiting examples of suitable sealable containers include pump-type bottles, nozzle-type bottles, tubes, sachets, packets, and various other configurations known to those skilled in the art.

An embodiment provides a composition for topical application, comprising:

a plurality of first core-shell microcapsules comprising a first core that comprises benzoyl peroxide and a first shell that comprises a first inorganic polymer; and

a plurality of second core-shell microcapsules comprising a second core that comprises a retinoid and a second shell that comprises a second inorganic polymer;

wherein the composition is an oil in water emulsion comprising a polyoxylstearate and a glycerylstearate wherein the ratio of the polyoxylstearate to the glycerylstearate is in the range of 0.1:10 to 10:0.1.

In various embodiments, the polyoxylstearate is selected from the group consisting of Polyoxyl-8 stearate, Polyoxyl-20 stearate, Polyoxyl-40 stearate, and Polyoxyl-100 stearate.

In various embodiments, the glycerylstearate is selected from the group consisting of glyceryl mono-stearate, glyceryl di-stearate and mixtures thereof.

In various embodiments, the amount of the polyoxylstearate in the composition is in the range of about 0.1% w/w to about 30% w/w.

In various embodiments, the amount of the glycerylstearate in the composition is in the range of about 0.1% w/w to about 30% w/w.

In various embodiments, the composition further comprises at least one fatty alcohol.

In various embodiments, the at least one fatty alcohol is selected from the group consisting of octyl alcohol, 2-ethyl hexanol, nonyl alcohol, decyl alcohol, undecanol, dodecyl alcohol, tridecyl alcohol, tetradecyl alcohol, pentadecyl alcohol, cetyl alcohol, palmitoleyl alcohol, heptadecyl alcohol, cetostearyl alcohol, stearyl alcohol, isostearyl alcohol, elaidyl alcohol, oleyl alcohol, linoleyl alcohol, elaidolinolenyl alcohol, ricinoleyl alcohol, nonadecyl alcohol, arachidyl alcohol, heneicosyl alcohol, behenyl alcohol, erucyl alcohol, lignoceryl alcohol, ceryl alcohol, montanyl alcohol, cluytyl alcohol, myricyl alcohol, melissyl alcohol, geddyl alcohol, cetearyl alcohol and mixtures thereof.

In various embodiments, the amount of the at least one fatty alcohol in the composition is in the range of about 0.2% w/w to about 50% w/w.

In various embodiments, the composition further comprises a polyacrylic acid homopolymer or copolymer.

In various embodiments, the oil in the oil in water emulsion is selected from the group consisting of paraffin oil, isopropyl myristate, caprylic/capric triglyceride, squalane, squalene, almond oil, castor oil, olive oil, jojoba oil, sunflower oil, soybean oil, grape seed oil, dimethicone, cyclomethicone and mixtures thereof.

In various embodiments, the oil in present in the composition in an amount in the range of about 0.05% w/w to about 50% w/w.

In various embodiments, the water in the oil in water emulsion further comprises at least one water soluble humectant.

In various embodiments, the at least one water soluble humectant is selected from the group consisting of propylene glycol, glycerin, and polyethylene glycol-X, where X is in the range of 200 to 10,000.

Another embodiment provides a composition for topical application, comprising:

a plurality of first core-shell microcapsules comprising a first core that comprises benzoyl peroxide and a first shell that comprises a first inorganic polymer; and

a plurality of second core-shell microcapsules comprising a second core that comprises a retinoid and a second shell that comprises a second inorganic polymer;

wherein the composition is in a gel form comprising at least one non-ionic polymeric dispersant and at least one thickening agent.

In various embodiments, the at least one non-ionic polymeric dispersant is selected from the group consisting of poly vinyl pyrrolidone (PVP), poly vinyl pyrrolidone-co-vinyl acetate, polyamide, polyurethane, polyurea and mixtures thereof.

In various embodiments, the at least one thickening agent is selected from the group consisting of hydroxy propyl cellulose (HPC), hydroxyl ethyl cellulose (HEC), hydroxyl methyl cellulose (HMC), polyacrylic acid homopolymer, polyacrylic acid copolymer, fatty alcohol, silica and its derivatives, xanthan gum, arabic gum, poly vinyl alcohol, veegum, laponite, clay, and mixtures thereof.

In various embodiments, the at least one thickening agent is a non-ionic agent.

In various embodiments, the at least one thickening agent is an ionic agent.

In various embodiments, the at least one thickening agent is present in the composition in an amount in the range of about 0.01% w/w to about 10% w/w.

In various embodiments, the composition further comprises glycerin.

In various embodiments, the non-ionic polymeric dispersant is present in the composition in an amount in the range of about 0.05% w/w to about 20% w/w.

In various embodiments, the composition further comprises at least one antimicrobial agent.

In various embodiments, the at least one antimicrobial agent is selected from the group consisting of methylparaben, propylparaben, phenoxyethanol, imidazolidinyl urea and mixtures thereof.

In various embodiments, at least one of the first core-shell microcapsules and the second core-shell microcapsules further comprise at least one phase changing material selected from the group consisting of a natural paraffin, a synthetic paraffin, an aliphatic alcohol, and a fatty acid.

In various embodiments, the first inorganic polymer is different from the second inorganic polymer.

In various embodiments, the first inorganic polymer and the second inorganic polymer are the same.

In various embodiments, the first inorganic polymer and the second inorganic polymer are each independently prepared from a sol-gel precursor selected from the group consisting of a metal alkoxide monomer, a semi-metal alkoxide monomer, a metal ester monomer, a semi-metal ester monomer, a silazane monomer, a colloidal silica, a monomer of the formula M(R)n(P)m, wherein M is a metallic or a semi-metallic element, R is a hydrolyzable substituent, n is an integer from 2 to 6, P is a non polymerizable substituent and m is an integer from 0 to 6, and a partially hydrolyzed and partially condensed polymer thereof.

In various embodiments, the semi-metal alkoxide monomer is selected from the group consisting of tetramethoxysilane, tetraethoxysilane, dimethyl dimethoxysilane, methyl trimethoxysilane, dimethyl dimethoxysilane, and sodium silicate.

In various embodiments, the amount of the benzoyl peroxide is in the range of about 1% to about 10% by weight, based on total weight of the composition.

In various embodiments, the amount of the retinoid is in the range of about 0.01% to about 1% by weight, based on total weight of the composition.

In various embodiments, the composition further comprises at least one antioxidant.

In various embodiments, the at least one antioxidant is selected from the group consisting of butylated hydroxytoluene (BHT), butylated hydroxyanisole, vitamin E, vitamin E acetate, vitamin E palmitate, vitamin C, an ester of vitamin C, and a salt of vitamin C or any combinations thereof.

In various embodiments, the at least one antioxidant is incorporated into the core of the second core-shell microcapsule.

An embodiment provides a composition for topical application, comprising:

a plurality of first core-shell microcapsules comprising a first core that comprises benzoyl peroxide and a first shell that comprises a first inorganic polymer;

a plurality of second core-shell microcapsules comprising a second core that comprises a retinoid and a second shell that comprises a second inorganic polymer; and

an amount of a stabilizing agent that is effective to:

(i) maintain the amount of benzoyl peroxide and the amount of retinoid in the composition at a level that is at least about 90% of the initial amounts, as measured after storage of the composition at a storage condition of 30° C. for three months; and

(ii) maintain the microbial count of the composition at a level that meets the criteria of USP 51, as measured by Antimicrobial Effectiveness Testing (AET) after storage of the composition at a storage condition of 40° C. for one month.

In various embodiments, the stabilizing agent is selected from the group consisting of polyvinylpyrrolidone, polyoxyl 100 stearate, glycerol monostearate, methylparaben, propylparaben, phenoxyethanol, imidazolidinyl urea and mixtures thereof.

In various embodiments, the stabilizing agent is further effective to (iii) maintain the bulk viscosity of the composition at about one million cps or less and within a range of about 70% to about 130% of the initial bulk viscosity, as measured after storage of the composition at 25° C. for three months.

In various embodiments, the bulk viscosity of the composition is maintained at about 500,000 cps or less.

In various embodiments, the stabilizing agent is further effective to (iv) maintain the microbial count of the composition at a level that meets the criteria of USP 61, as measured by Microbial Limits Testing (MLT) after storage of the composition at a storage condition of 25° C. for six months.

In various embodiments, the stabilizing agent is present in an amount effective to maintain the amount of benzoyl peroxide and the amount of retinoid in the composition at a level of at least about 90% of the initial amounts, as measured after storage of the composition at a storage condition selected from the group consisting of 40° C. for two months, 25° C. for six months and 5° C. for two years.

In various embodiments, the stabilizing agent is present in an amount effective to maintain the amount of benzoyl peroxide and the amount of retinoid in the composition at a level of at least about 97% of the initial amounts, as measured after storage of the composition at a storage condition of 5° C. for two years.

In various embodiments, the stabilizing agent is a mixture comprising at least two selected from the group consisting of polyvinylpyrrolidone, polyoxyl 100 stearate, glycerol monostearate, methylparaben, propylparaben, phenoxyethanol, and imidazolidinyl urea.

In various embodiments, the second core further comprises at least one phase changing material selected from the group consisting of a natural paraffin, a synthetic paraffin, an aliphatic alcohol, and a fatty acid.

In various embodiments, the first inorganic polymer is different from the second inorganic polymer.

In various embodiments, the first inorganic polymer and the second inorganic polymer are the same.

In various embodiments, the first inorganic polymer and the second inorganic polymer are each independently prepared from a sol-gel precursor selected from the group consisting of a metal alkoxide monomer, a semi-metal alkoxide monomer, a metal ester monomer, a semi-metal ester monomer, a silazane monomer, a colloidal silica, a monomer of the formula M(R)ₙ(P)ₘ, wherein M is a metallic or a semi-metallic element, R is a hydrolyzable substituent, n is an integer from 2 to 6, P is a non polymerizable substituent and m is an integer from 0 to 6, and a partially hydrolyzed and partially condensed polymer thereof.

In various embodiments, the semi-metal alkoxide monomer is selected from the group consisting of tetramethoxysilane, tetraethoxysilane, dimethyl dimethoxysilane, methyl trimethoxysilane, dimethyl dimethoxysilane, and sodium silicate.

In various embodiments, the amount of the benzoyl peroxide is in the range of about 1% to about 10% by weight, based on total weight of the composition.

In various embodiments, the amount of the retinoid (ATRA) is in the range of about 0.01% to about 0.1% by weight, based on total weight of the composition.

In various embodiments, the composition is in a form selected from the group consisting of an emulsion, a cream, an aqueous solution, an oil, an ointment, a paste, a gel, a lotion, and a suspension.

In various embodiments, the microcapsules are configured to release at least one of the benzoyl peroxide and the retinoid by extraction.

In various embodiments, the second core further comprises an antioxidant.

In various embodiments, the antioxidant is selected from the group consisting of butylated hydroxytoluene (BHT), butylated hydroxyanisole, vitamin E, vitamin E acetate, vitamin E palmitate, vitamin C, an ester of vitamin C, and a salt of vitamin C.

Another embodiment provides a composition in a gel form for topical application, comprising:

a plurality of first core-shell microcapsules comprising a first core that comprises benzoyl peroxide and a first shell that comprises a first silica polymer;

a plurality of second core-shell microcapsules comprising a second core that comprises all-trans retinoic acid (ATRA) and a second shell that comprises a second silica polymer; and

at least one non-ionic polymer in an amount effective to provide at least one stabilization selected from the group consisting of

(i) viscosity stabilization whereby the viscosity of the composition is maintained such that the change in viscosity of the composition, as measured after manufacture followed by three months storage at 25° C., is less than about 30%, and

(ii) degradation stabilization whereby the amount of degradation of the ATRA, as measured after manufacture followed by three months storage at 25° C., is less than about 10%.

In various embodiments, the non-ionic polymer is present in an amount effective to provide the viscosity stabilization.

In various embodiments, the viscosity stabilization is effective to maintain the viscosity of the composition at more than about 25,000 cps as measured after manufacture followed by three months storage at 25° C.

In various embodiments, the non-ionic polymer is present in an amount effective to provide the degradation stabilization.

In various embodiments, the amount of degradation of the ATRA is less than about 5%.

In various embodiments, the non-ionic polymer is present in an amount effective to provide the viscosity stabilization and the degradation stabilization.

In various embodiments, the non-ionic polymer is polyvinylpyrrolidone.

In various embodiments, the second core further comprises at least one phase changing material selected from the group consisting of a natural paraffin, a synthetic paraffin, an aliphatic alcohol, a fatty acid, an ester of an aliphatic alcohol, and an ester of a fatty acid.

In various embodiments, the ester of a fatty acid comprises a beeswax.

In various embodiments, the second core further comprises an antioxidant.

In various embodiments, the antioxidant is selected from the group consisting of butylated hydroxytoluene (BHT), butylated hydroxyanisole, vitamin E, vitamin E acetate, vitamin E palmitate, vitamin C, an ester of vitamin C, and a salt of vitamin C.

In various embodiments, the first inorganic polymer is different from the second inorganic polymer.

In various embodiments, the first inorganic polymer and the second inorganic polymer are the same.

In various embodiments, the first silica polymer and the second silica polymer are each independently prepared from a sol-gel precursor selected from the group consisting of tetramethoxysilane, tetraethoxysilane, and sodium silicate.

In various embodiments, the initial amount of the benzoyl peroxide is in the range of about 1% to about 10% by weight, based on the total weight of the composition.

In various embodiments, the initial amount of the benzoyl peroxide is about 6% by weight, based on the total weight of the composition.

In various embodiments, the initial amount of the benzoyl peroxide is about 3% by weight, based on the total weight of the composition.

In various embodiments, the initial amount of the ATRA is in the range of about 0.01% to about 0.1% by weight, based on the total weight of the composition.

In various embodiments, the initial amount of the ATRA is about 0.1% by weight, based on the total weight of the composition.

In various embodiments, the initial amount of the ATRA is about 0.05% by weight, based on the total weight of the composition.

Another embodiment provides a composition in an emulsion form for topical application, comprising:

a plurality of first core-shell microcapsules comprising a first core that comprises benzoyl peroxide and a first shell that comprises a first silica polymer;

a plurality of second core-shell microcapsules comprising a second core that comprises all-trans retinoic acid (ATRA) and a second shell that comprises a second silica polymer; and

a stabilizing agent comprising at least one non-ionic surfactant and at least one antimicrobial in amounts effective to:

(i) maintain the amount of benzoyl peroxide and the amount of retinoid in the composition at a level that is at least about 90% of the initial amounts, as measured after storage of the composition at a storage condition selected from the group consisting of 40° C. for two months, 30° C. for three months, 25° C. for six months and 5° C. for two years; and

(ii) maintain the microbial count of the composition at a level that meets the criteria of USP 51, as measured by Antimicrobial Effectiveness Testing (AET) after storage of the composition at a storage condition of 40° C. for one month.

In various embodiments, the stabilizing agent is further effective to (iii) maintain the bulk viscosity of the composition at about three million cps or less and within a range of about 70% to about 130% of the initial bulk viscosity, as measured after storage of the composition at 25° C. for three months.

In various embodiments, the stabilizing agent is effective to maintain the bulk viscosity of the composition in the range of about 80000 cps to about one million cps as measured after storage of the composition at 25° C. for three months.

In various embodiments, the stabilizing agent is further effective to (iv) maintain the microbial count of the composition at a level that meets the criteria of USP 61, as measured by Microbial Limits Testing (MLT) after storage of the composition at a storage condition of 25° C. for six months.

In various embodiments, the stabilizing agent is present in an amount effective to maintain the amount of benzoyl peroxide and the amount of retinoid in the composition at a level of at least about 97% of the initial amounts, as measured after storage of the composition at a storage condition of 5° C. for two years.

In various embodiments, the non-ionic surfactant comprises polyoxyl 100 stearate, glycerol monostearate or a combination thereof.

In various embodiments, the second core further comprises at least one phase changing material selected from the group consisting of a natural paraffin, a synthetic paraffin, an aliphatic alcohol, a fatty acid, an ester of an aliphatic alcohol, and an ester of a fatty acid.

In various embodiments, the ester of a fatty acid comprises a beeswax.

In various embodiments, the second core further comprises an antioxidant.

In various embodiments, the antioxidant is selected from the group consisting of butylated hydroxytoluene (BHT), butylated hydroxyanisole, vitamin E, vitamin E acetate, vitamin E palmitate, vitamin C, an ester of vitamin C, and a salt of vitamin C.

In various embodiments, the first inorganic polymer is different from the second inorganic polymer.

In various embodiments, the first inorganic polymer and the second inorganic polymer are the same.

In various embodiments, the first inorganic polymer and the second inorganic polymer are each independently prepared from a sol-gel precursor selected from the group consisting of tetramethoxysilane, tetraethoxysilane, and sodium silicate.

In various embodiments, the initial amount of the benzoyl peroxide is in the range of about 1% to about 10% by weight, based on the total weight of the composition.

In various embodiments, the initial amount of the benzoyl peroxide is about 6% by weight, based on the total weight of the composition.

In various embodiments, the initial amount of the benzoyl peroxide is about 3% by weight, based on the total weight of the composition.

In various embodiments, the initial amount of the ATRA is in the range of about 0.01% to about 0.1% by weight, based on the total weight of the composition.

In various embodiments, the initial amount of the ATRA is about 0.1% by weight, based on the total weight of the composition.

In various embodiments, the initial amount of the ATRA is about 0.05% by weight, based on the total weight of the composition.

Another embodiment provides a composition in an emulsion form for topical application, comprising:

a plurality of first core-shell microcapsules comprising a first core that comprises benzoyl peroxide and a first shell that comprises a first silica polymer, the benzoyl peroxide being present in the composition in an initial amount of about 6% by weight, based on the total weight of the composition;

a plurality of second core-shell microcapsules comprising a second core that comprises all-trans retinoic acid (ATRA) and a second shell that comprises a second silica polymer, the ATRA being present in the composition in an initial amount of about 0.1% by weight, based on the total weight of the composition; and

a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea;

wherein the stabilizing agent is present in an amount effective to:

(ia) maintain the amount of benzoyl peroxide at a level of at least about 5.4% by weight based on the total weight of the composition, as measured after storage of the composition at a storage condition selected from the group consisting of 40° C. for two months, 30° C. for three months, 25° C. for six months and 5° C. for two years;

(ib) maintain the amount of ATRA at a level of at least about 0.09% by weight based on the total weight of the composition, as measured after storage of the composition at a storage condition selected from the group consisting of 40° C. for two months, 30° C. for three months, 25° C. for six months and 5° C. for two years;

(ii) maintain the microbial count of the composition at a level that meets the criteria of USP 51, as measured by Antimicrobial Effectiveness Testing (AET) after storage of the composition at a storage condition of 40° C. for one month; and

(iii) maintain the bulk viscosity of the composition at about one million cps or less and within a range of about 70% to about 130% of the initial bulk viscosity, as measured after storage of the composition at 25° C. for three months.

In various embodiments, the stabilizing agent is further effective to (iv) maintain the microbial count of the composition at a level that meets the criteria of USP 61, as measured by Microbial Limits Testing (MLT) after storage of the composition at a storage condition of 25° C. for six months.

Another embodiment provides a composition in an emulsion form for topical application, comprising:

a plurality of first core-shell microcapsules comprising a first core that comprises benzoyl peroxide and a first shell that comprises a first silica polymer, the benzoyl peroxide being present in the composition in an initial amount of about 3% by weight, based on the total weight of the composition;

a plurality of second core-shell microcapsules comprising a second core that comprises all-trans retinoic acid (ATRA) and a second shell that comprises a second silica polymer, the ATRA being present in the composition in an initial amount of about 0.05% by weight, based on the total weight of the composition; and

a stabilizing agent comprising (a) at least one non-ionic surfactant selected from the group consisting of polyoxyl 100 stearate and glycerol monostearate; and (b) at least one antimicrobial selected from the group consisting of methylparaben and imidazolidinyl urea;

wherein the stabilizing agent is present in an amount effective to:

(ia) maintain the amount of benzoyl peroxide at a level of at least about 2.7% by weight based on the total weight of the composition, as measured after storage of the composition at a storage condition selected from the group consisting of 40° C. for two months, 30° C. for three months, 25° C. for six months and 5° C. for two years;

(ib) maintain the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years;

(ii) maintain the microbial count of the composition at a level that meets the criteria of USP 51, as measured by Antimicrobial Effectiveness Testing (AET) after storage of the composition at a storage condition of 40° C. for one month; and

(iii) maintain the bulk viscosity of the composition at about one million cps or less and within a range of about 70% to about 130% of the initial bulk viscosity, as measured after storage of the composition at 25° C. for three months.

In various embodiments, the stabilizing agent is further effective to (iv) maintain the microbial count of the composition at a level that meets the criteria of USP 61, as measured by Microbial Limits Testing (MLT) after storage of the composition at a storage condition of 25° C. for six months.

Another embodiment provides a packaged product, comprising a sealable container and the composition as described herein contained within the sealable container.

In one aspect, the present invention provides a method of treatment of acne in a patient in need thereof, comprising the concomitant topical administration once daily for a period of up to 3 months of a stable composition, comprising from about 2% w/w to about 10% w/w of solid particulate benzoyl peroxide (BPO) and from about 0.01% w/w to about 0.1% w/w of solid particulate all trans retinoic acid (ATRA), wherein 90% of the solid particles of ATRA are in the range of 5-20 microns, wherein the side-effects of the two active agents are medically acceptable and wherein the therapeutic effect of BPO and ATRA is superior to the effect of each active agent administered alone.

In some embodiments the stable composition comprises from about 2% w/w to about 10% w/w of solid particulate benzoyl peroxide (BPO) and from about 0.01% w/w to about 0.1% w/w of solid particulate all trans retinoic acid (ATRA) wherein none of the two active agents BPO and ATRA is encapsulated.

In some embodiments, the stable composition comprises from about 2% w/w to about 10% w/w of solid particulate benzoyl peroxide (BPO) and from about 0.01% w/w to about 0.1% w/w of solid particulate all trans retinoic acid (ATRA), wherein one of the two active agents BPO and ATRA is encapsulated.

In some embodiments, the stable composition, comprises from about 2% w/w to about 10% w/w of solid particulate benzoyl peroxide (BPO) and from about 0.01% w/w to about 0.1% w/w of solid particulate all trans retinoic acid (ATRA), wherein both actives BPO and ATRA are encapsulated either partially or entirely.

In certain embodiments, the stable composition as used according to the method of treatment of acne of the present invention comprises a plurality of first core-shell microcapsules comprising a first core that comprises BPO and a first shell that comprises a first inorganic polymer; and a plurality of second core-shell microcapsules comprising a second core that comprises ATRA and a second shell that comprises a second inorganic polymer; wherein the composition is an oil in water emulsion comprising a polyoxylstearate and a glycerylstearate, wherein the ratio of said polyoxylstearate to said glycerylstearate is in the range of 0.1:10 to 10:0.1.

In some particular embodiments, the stable composition comprises an initial concentration of 3% w/w BPO and an initial concentration of 0.05% w/w ATRA, wherein the composition maintains the amount of benzoyl peroxide at a level of at least about 2.7% by weight based on the total weight of the composition, as measured after storage of the composition at a storage condition selected from the group consisting of 40° C. for two months, 30° C. for three months, 25° C. for six months and 5° C. for two years and maintains the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years; (ii) maintain the microbial count of the composition at a level that meets the criteria of USP 51, as measured by Antimicrobial Effectiveness Testing (AET) after storage of the composition at a storage condition of 40° C. for one month; and maintains the bulk viscosity of said composition at about one million cps or less and within a range of about 70% to about 130% of the initial bulk viscosity, as measured after storage of the composition at 25° C. for three months.

According to some embodiments, the concentration of ATRA in the stable compositions is from about 0.04% w/w to about 0.1% w/w, preferably about 0.1% w/w, more preferably about 0.08% w/w and even more preferably about 0.05% w/w or 0.04% w/w.

In some embodiments, the stable composition comprises from about 2% w/w to about 10% w/w BPO, preferably from about 2.5% w/w to about 5% w/w, preferably about 3% w/w or about 3.75% w/w or about 6.28% w/w.

In some embodiments, the stable composition comprises 3% w/w BPO and from about 0.05% w/w to about 0.1% w/w ATRA.

In some embodiments, the stable BPO/ATRA composition is administered for up to 2 weeks, up to 1 month, preferably up to 2 months and more preferably up to 3 months.

In some embodiments, in the method of the present invention the concomitant administration of BPO and ATRA is carried out by once daily topical administration of a single composition comprising both BPO and ATRA.

In an embodiment, there is provided a method of treatment comprising concomitant administration carried out by topical administration of a first composition comprising from about 2% w/w to about 10% w/w BPO, and a second composition comprising from about 0.1% w/w to about 0.2% w/w ATRA, concomitantly administered once daily from a dual chamber dispenser or from two separate dispensers and mixed before applying on the skin of a patient in need thereof, thus administering a combination product comprising from about 1% w/w to about 5% w/w BPO and from about 0.05% w/w to about 0.1% w/w ATRA on the skin of said patient.

In another embodiment, there is provided a method of treatment comprising the concomitant topical administration of a first composition comprising 6% w/w BPO, and a second composition comprising 0.1% w/w ATRA, concomitantly administered once daily for a period of up to 3 months from a dual chamber dispenser or from two separate dispensers and mixed before applying on the skin of a patient in need thereof, thus administering a combination product comprising 3% BPO and 0.05% ATRA on the skin of said patient.

In yet another embodiment, there is provided a method of treatment, wherein the first composition comprises a plurality of first core-shell microcapsules comprising a first core that comprises BPO and a first shell that comprises a first inorganic polymer and the second composition comprises a plurality of second core-shell microcapsules comprising a second core that comprises ATRA and a second shell that comprises a second inorganic polymer; wherein said composition is an oil in water emulsion comprising a polyoxylstearate and a glycerylstearate wherein the ratio of said polyoxylstearate to said glycerylstearate is in the range of 0.1:10 to 10:0.1.

In another aspect, the present invention provides a dual chamber dispenser (for example 2×30 ml) having a first chamber charged with a first composition comprising from about 2% w/w to about 10% w/w BPO, and the second chamber charged with a second composition comprising from about 0.01% w/w to about 0.2% w/w ATRA, wherein the two compositions are mixed before applying on the skin of a patient in need thereof.

In yet another aspect, there is provided a dual chamber dispenser with a single actuator and two nozzles, having a first chamber charged with the first composition of claim 12 comprising 6% w/w BPO, and the second chamber charged with the second composition of claim 12 comprising 0.1% w/w ATRA wherein the two compositions are mixed before applying on the skin of a patient in need thereof, thus administering a combination product comprising 3% BPO and 0.05% ATRA on the skin of said patient.

In a further aspect, the present invention provides a regimen of administration comprising daily administration to a patient in need thereof of a stable composition comprising from about 2% w/w to about 10% w/w of solid particulate benzoyl peroxide (BPO) and from about 0.01% w/w to about 0.1% w/w of solid particulate all trans retinoic acid (ATRA) for up to 2 weeks, up to 1 month, preferably up to 2 months and more preferably up to 3 months.

In yet another aspect, the present invention provides a regimen of administration comprising daily administration to a patient in need thereof of a first composition comprising from about 2% w/w to about 10% w/w BPO, and a second composition comprising from about 0.01% w/w to about 0.2% w/w ATRA, wherein the two compositions are concomitantly administered once daily from a dual chamber dispenser or from two separate dispensers and mixed before applying on the skin of a patient in need thereof for up to 2 weeks, up to 1 month, preferably up to 2 months and more preferably up to 3 months.

### EXAMPLES

Additional embodiments are disclosed in further detail in the following examples, which are not in any way intended to limit the scope of the claims.

### Example 1. Preparation of Encapsulated ATRA (3.06% E-ATRA Water Suspension)

### a) Oil Phase

8.62 grams of Butylated hydroxyl toluene (BHT) and 45.9 grams of all-trans retinoic acid (ATRA) were mixed in 129.3 grams of Squalane. 86.16 grams of Tetroethoxysilane (TEOS) were added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled tretinoin in oil was aliquoted out and then heated to 60° C. 9.0 grams of Beeswax were added and melted in the oil phase.

### b) Water Phase

3.3 grams CTAC (Cetrimonium Chloride) were dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) was added to the water phase and homogenized at 4000 rpm for 1 minute. 17.9 grams of sodium silicate extra pure solution (28%) were added to the emulsion. The pH of the emulsion was adjusted to 4.0 using HCl 5N solution. Water was added to complete the total weight of the mixture to 650 grams. The suspension was then stirred for 17 hours at 25° C. for the TEOS hydrolysis to be completed. The composition of the final encapsulated ATRA water suspension product is shown in Table 1.

TABLE 1
Composition of the encapsulated ATRA 3.06% water suspension

| | % of pure ingredient in the |
|---|---|
| Ingredient | suspension |
| Beeswax | 1.15 |
| Squalane | 8.62 |
| TEOS | 5.74 |
| ATRA | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Example 2. Preparation of Encapsulated BPO (15% E-BPO Water Suspension)

### a) Preparation of Benzoyl Peraxide Solution and Acid Cocktail

A benzoyl peroxide (BPO) solution was prepared by mixing 125.67 grams of CTAC CT-429 (Cetrimonium Chloride 30%), 3008 grams of hydrous benzoyl peroxide, and 5200 g water under high shear. The solution was homogenized for 60 minutes at 33° C. (no more than 45° C.), and then the pH of the solution was adjusted to 7.0 using sodium hydroxide solution (20%).

An acid cocktail was prepared using 493 grams Hydrochloric acid (37%), 98 grams anhydrous Citric Acid, 147 grams Lactic Acid (90%), and 794 grams water.

### b) Coating Cycle

The coating cycle was started by adding 38 grams sodium silicate solution extra pure (28%) to the benzoyl peroxide solution prepared in step a) under high shear, followed by adding the acid cocktail prepared in step (a) to adjust the pH to be lower than 6.8, and followed by adding 57 grams PDAC (3%) solution to the mixture. The cycle was repeated 50 times while the mixture was stirred under high shear for 17 hours. After the 50 cycles, the pH of the mixture was adjusted to 5.0 using the acid cocktail, and water was added to complete the total weight of the mixture to 15 kilograms. The composition of the final BPO water suspension product is shown in Table 2.

TABLE 2
Composition of the encapsulated BPO 15% water suspension

| | % of ingredient in the |
|---|---|
| Ingredient | suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Hydrous Benzoyl Peroxide | 15.00 |
| Sterile Water for Irrigation | 78.83 |

### Example 3. Preparation of Formulation of Encapsulated ATRA (0.1%) and Encapsulated BPO (6%) in Emulsion (Formulation I)

Oil Phase: 720.0 of grams Cyclomethicone 5-N, 540.0 of grams Cetyl Alcohol, 360.0 grams Polyoxyl 100 Stearate, and 540.0 grams of Glyceryl Monosterate were mixed at 70° C.

Water phase: 18.0 grams of Ethylendiaminetetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 7152 grams of encapsulated BPO 15% water suspension made as described in Example 2 were mixed. The resulting mixture was added to the emulsion at 65° C. and mixed at 1400 rpm for 10 minutes. The emulsion was cooled to 35° C. and the pH of the emulsion was adjusted to 3.5 using HCl 5N solution. After 588.2 grams of encapsulated ATRA 3.06% water suspension made as described in Example 1 were added, the emulsion was stirred at 1400 rpm for 10 minutes. HCl 5N was added to adjust the pH to 3.6, and then water was added until the total weight of the emulsion reached 18 kilograms. The composition of the formulation prepared in this example is shown in Table 3.

TABLE 3
Composition of Formulation I

| | % of pure ingredient in the |
|---|---|
| Ingredient | composition |
| Polyquarternium-7 | 0.21 |
| Hydrochloric Acid | 0.51 |
| Citric Acid, Anhydrous | 0.58 |
| Lactic Acid | 0.25 |
| Silicon Dioxide | 1.44 |
| Sodium hydroxide | 0.09 |
| Cetrimonium Chloride | 0.11 |
| Hydrous Benzoyl Peroxide | 6.00 |
| Beeswax | 0.04 |
| Squalane | 0.28 |
| Ethanol (Alcohol) | 0.14 |
| ATRA | 0.10 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | 73.72 |

### Example 4. Preparation of Formulation of Encapsulated ATRA (0.1%) and Encapsulated BPO (6%) in Gel (Formulation II)

20.0 grams of PVP (Plasdone K-29/30) and 80 grams of water were mixed to prepare 100 grams of 20% poly vinylpyrrolidone (PVP) solution. 850.0 grams of Glycerin (99.5%) and 7900 grams of water were mixed and stirred for 10 minutes at 2800 rpm. 212.5 grams of Natrosol (250HHX) and 42.5 grams of Klucel (HF Pharm) were added, and the resulted gel was homogenized at 8400 rpm for at least 70 minutes until it was free of lumps. After 17.0 grams of PVP (20%) and 6755 grams of encapsulated BPO 15% water suspension prepared as described in Example 2 were mixed and stirred for 10 minutes, the mixture was added to the gel under high shear. The pH of the gel was then adjusted to 3.5 using HCl 5N. After 6.9 grams of PVP (20%) and 555.55 grams of encapsulated ATRA 3.06% water suspension prepared as described in Example 1 were mixed and stirred for 10 minutes, the mixture was added to the gel and the pH of the gel was then adjusted to 3.5 using HCl 5N. Water was added to complete the total weight of the mixture to 17 kilograms, and the mixture was finally mixed until homogeneity. The composition of the formulation prepared in this example is shown in Table 4.

TABLE 4
Composition of Formulation II

| | % of pure ingredient in the |
|---|---|
| Ingredient | composition |
| Polyquarternium-7 | 0.21 |
| Hydrochloric Acid | 0.59 |
| Citric Acid, Anhydrous | 0.18 |
| Lactic Acid | 0.25 |
| Silicon Dioxide | 1.44 |
| Cetrimonium Chloride | 0.11 |
| Hydrous Benzoyl Peroxide | 6.00 |
| Beeswax | 0.04 |
| Squalane | 0.28 |
| Ethanol (Alcohol) | 0.14 |
| ATRA | 0.10 |
| Butylated hydroxytoluene | 0.02 |
| Glycerin | 5.00 |
| Hydroxypropyl cellulose | 0.25 |
| Hydroxyethyl cellulose | 1.25 |
| Povidone K-30 (PVP) | 0.03 |
| Sterile Water for Irrigation | 84.32 |

### Example 5. Preparation of Encapsulated ATRA (0.635% E-ATRA Water Suspension)

### a) Oil Phase

8.62 grams of Butylated hydroxyl toluene (BHT) and 29.7 grams of all-trans retinoic acid (ATRA) were mixed in 145.51 grams of Squalane. 86.17 grams of Tetroethoxysilane (TEOS) were added, and the resulted mixture was milled in a ball mill at 5000 rpm for 10 minutes at 25° C. with an upper propeller mixer at a speed of 250 rpm for 10 minutes. 196.56 grams of milled tretinoin in oil was aliquoted out and then heated to 60° C. 12.6 grams Beeswax were added and melted in the oil phase.

### b) Water Phase

5.28 grams of CTAC (Cetrimonium Chloride) were dissolved in 792.0 g of water at 60° C.

### c) Core-Shell Step

199.2 grams of the oil phase prepared in step (a) was added to the water phase and homogenized at 4000 rpm for 1 minute. 28.64 grams of Sodium Silicate extra pure solution (28%) were added to the emulsion. The pH of the emulsion was adjusted to 4.1 using HCl 5N solution. The emulsion was stirred for 17 hours at 25° C., and the pH of the emulsion was at 4.2.

### d) Coating Step

960.0 grams of encapsulated ATRA made in step (c) were added to 945.0 grams of water under high shear. 7.68 grams of Sodium Silicate extra pure solution (28%) were added, the pH of the mixture was then adjusted to 2.3 using HCl 5N solution. The coating cycle was repeated for 10 times. After a total of 76.8 grams of Sodium Silicate (25%) was added, 947.2 grams of PDAC-7 (3%) was added and the mixture was homogenized for 10 minutes. The pH of the mixture was adjusted to 4.3 using Sodium Hydroxide 10% solution, and water was added to complete the total weight of the mixture to 2992 grams. The composition of the final ATRA water suspension product is shown in Table 5.

TABLE 5
Composition of the encapsulated ATRA 0.635% water suspension

| | % of pure ingredient in the |
|---|---|
| Ingredient | suspension |
| Beeswax | 0.36 |
| Squalane | 3.11 |
| TEOS | 1.84 |
| ATRA | 0.64 |
| Cetrimonium Chloride | 0.05 |
| Silicon Dioxide | 0.93 |
| Hydrochloric acid | 0.29 |
| Polyquantrium-7 | 0.90 |
| Butylated hydroxytoluene | 0.18 |
| Sterile Water for Irrigation | 91.88 |

### Example 6. Preparation of Formulations of Encapsulated ATRA (0.1%) and Encapsulated BPO (6%) in Gel (Formulation III)

850.0 grams of Glycerin (99.5%) and 4600 grams of water were mixed and stirred for 10 minutes at 2800 rpm. After 212.5 grams of Natrosol (250HHX) and 42.5 grams of Klucel (HF Pharm) were added sequentially, the resulted gel was homogenized at 8400 rpm for at least 70 minutes until it was free of lumps. 750.0 grams Polyquarternium-7 (10%) were added to 1750.0 grams water to prepare 3% PDAC solution. After 1133.9 grams of 3% PDAC solution and 6666.7 grams of encapsulated BPO 15% water suspension were mixed, the mixture was added to the gel under high shear. The pH of the gel was then adjusted to 3.5 using HCl 5N solution. After 2672.9 grams of encapsulated ATRA 0.635% water suspension prepared according to the procedure described in Example 5 were added, the gel was stirred at 1400 rpm for 15 minutes, and the pH of the gel was then adjusted to 3.6 using HCl 5N solution. After water was added to complete the total weight to 17 kilograms, the mixture was finally mixed until homogeneity. The composition of the formulation prepared in this example is shown in Table 6.

TABLE 6
Composition of Formulation III

| | % of pure ingredient in the |
|---|---|
| Ingredient | composition |
| Polyquarternium-7 | 0.54 |
| Hydrochloric Acid | 0.55 |
| Citric Acid, Anhydrous | 0.18 |
| Lactic Acid | 0.25 |
| Silicon Dioxide | 1.59 |
| Cetrimonium Chloride | 0.11 |
| Hydrous Benzoyl Peroxide | 6.00 |
| Beeswax | 0.06 |
| Squalane | 0.49 |
| Ethanol (Alcohol) | 0.22 |
| ATRA | 0.10 |
| Butylated hydroxytoluene | 0.03 |
| Glycerin | 5.00 |
| Hydroxypropyl cellulose | 0.25 |
| Hydroxyethyl cellulose | 1.25 |
| Sterile Water for Irrigation | 83.54 |

### Example 7. Preparation of Formulations of Encapsulated ATRA (0.1%) and Encapsulated BPO (6%) in Gel (Formulations IV-VII)

Four additional formulations of encapsulated ATRA and encapsulated BPO were prepared following the procedures as described in the Example section of the International Patent Application PCT/IL2010/001092. The compositions of each of the four formulations prepared in this example are shown in Tables 7-10.

TABLE 7
Composition of Formulation IV

| | % of pure ingredient in the |
|---|---|
| Ingredient | composition |
| Polyquarternium-7 | 0.22 |
| Hydrochloric Acid | 0.40 |
| Citric Acid, Anhydrous | 0.18 |
| Lactic Acid | 0.25 |
| Silicon Dioxide | 1.61 |
| Sodium hydroxide | 0.16 |
| Cetrimonium Chloride | 0.11 |
| Hydrous Benzoyl Peroxide | 6.00 |
| Beeswax | 0.06 |
| Squalane | 0.49 |
| Ethanol (Alcohol) | 0.19 |
| ATRA | 0.10 |
| Butylated hydroxytoluene | 0.03 |
| Carbomer 980 | 1.00 |
| Hydroxyethyl cellulose | 0.70 |
| Sterile Water for Irrigation | 88.39 |

TABLE 8
Composition of Formulation V

| | % of pure ingredient in the |
|---|---|
| Ingredient | composition |
| Polyquarternium-7 | 0.22 |
| Hydrochloric Acid | 0.40 |
| Citric Acid, Anhydrous | 0.18 |
| Lactic Acid | 0.25 |
| Silicon Dioxide | 1.61 |
| Sodium hydroxide | 0.28 |
| Cetrimonium Chloride | 0.11 |
| Hydrous Benzoyl Peroxide | 6.00 |
| Beeswax | 0.06 |
| Squalane | 0.49 |
| Ethanol (Alcohol) | 0.19 |
| ATRA | 0.10 |
| Butylated hydroxytoluene | 0.03 |
| Carbomer 980 | 1.20 |
| Carbomer 1342 | 0.30 |
| Sterile Water for Irrigation | 88.47 |

TABLE 9
Composition of Formulation VI

| | % of pure ingredient in the |
|---|---|
| Ingredient | composition |
| Polyquarternium-7 | 0.22 |
| Hydrochloric Acid | 0.56 |
| Citric Acid, Anhydrous | 0.18 |
| Lactic Acid | 0.25 |
| Silicon Dioxide | 1.61 |
| Sodium hydroxide | 0.01 |
| Cetrimonium Chloride | 0.11 |
| Hydrous Benzoyl Peroxide | 6.00 |
| Beeswax | 0.06 |
| Squalane | 0.49 |
| Ethanol (Alcohol) | 0.19 |
| ATRA | 0.10 |
| Butylated hydroxytoluene | 0.03 |
| Glycerin | 15.00 |
| Hydroxypropyl cellulose | 0.50 |
| Hydroxyethyl cellulose | 1.25 |
| Sterile Water for Irrigation | 73.34 |

TABLE 10
Composition of Formulation VII

| | % of pure ingredient in the |
|---|---|
| Ingredient | composition |
| Polyquarternium-7 | 0.56 |
| Hydrochloric Acid | 0.56 |
| Citric Acid, Anhydrous | 0.18 |
| Lactic Acid | 0.25 |
| Silicon Dioxide | 1.61 |
| Sodium hydroxide | 0.01 |
| Cetrimonium Chloride | 0.11 |
| Hydrous Benzoyl Peroxide | 6.00 |
| Beeswax | 0.06 |
| Squalane | 0.49 |
| Ethanol (Alcohol) | 0.19 |
| ATRA | 0.10 |
| Butylated hydroxytoluene | 0.03 |
| Glycerin | 5.00 |
| Hydroxypropyl cellulose | 0.25 |
| Hydroxyethyl cellulose | 1.25 |
| Sterile Water for Irrigation | 83.25 |

### Example 8. Stability of Formulations of Encapsulated ATRA and Encapsulated BPO

In this example, formulations that were prepared according to the procedures described in Examples 3, 4, 6 and 7 were stored at 5° C. and 25° C./60% RH, respectively, for three months to evaluate the stability of ATRA and BPO.

### ATRA Stability Analysis

Diluent was prepared by dissolving 1 g of BHT in 1000 ml of acetonitrile. ATRA and its degradation products were extracted from each of the formulations with the diluent. The content of ATRA and its unknown degradation products were determined in comparison with an external standard by HPLC method using Agilent 1200 HPLC system or equivalent. The HPLC conditions used are shown in Table 11.

TABLE 11
HPLC Conditions for ATRA Content Analysis

| Column | Zorbax RX-C18 3.5 μm 4.6*150 mm | | | |
|---|---|---|---|---|
| Mobile phase | Eluent A - acetonitrile | | | |
| | Eluent B- 1% acetic acid in water | | | |
| | Eluent C - methanol | | | |
| Gradient program | Time | % A | % B | % C |
| | 5 | 50 | 40 | 10 |
| | 25 | 90 | 0 | 10 |
| | 26 | 50 | 40 | 10 |
| | 35 | 50 | 40 | 10 |

| | |
|---|---|
| Flow rate | 1.3 ml/min |
| Detection | UV, wavelength 330 nm |
| Injection volume | 10 μL |
| Column temperature | 35° C. |
| Auto-sampler temperature | 4° C. |

The ATRA content in the sample was calculated using the formula:

$$\% ATRA = \frac{Asample * V * P}{Rf * Wsample}$$

where:
Aₛₐₘₚₗₑ=ATRA peak area arising from the Sample Preparation;
Rf=averaged response factor (area/concentration) (average of five injections of first standard and one injection of second standard);
Wsampe=sample weight in mg;
V=sample solution volume (50 ml);
P=ATRA standard purity in percentage.

The content of individual degradation product (e.g., RRT 0.248, 0.538, 0.560) in the sample as percentage from labeled amount of ATRA (0.1%) was calculated using the formula:

$$\% degradation product = \frac{Adp * V * P * 100}{Rf * Wsample * 0.1}$$

where:
A_{dp}=degradation product peak area arising from the Sample Preparation;
Rf=averaged response factor (area/concentration) (average of five injections of first standard and one injection of second standard);
Wₛₐₘₚₗₑ=sample weight in mg;
V=sample solution volume (50 ml);
P=ATRA standard purity in percentage.

The sum of other unknown degradation products in the sample as percentage from labeled amount of ATRA (0.1%) was calculated using the formula:

$$\% Sum of other degradation product = \frac{Σ Adp * V * P * 100}{Rf * Wsample * 0.1}$$

where:
ΣA_{dp}=sum of area of other unknown degradation product arising from the Sample Preparation;
Rf=averaged response factor (area/concentration) (average of five injections of first standard and one injection of second standard);
Wₛₐₘₚₗₑ=sample weight in mg;
V=sample solution volume (50 ml);
P=ATRA standard purity in percentage.

A summary of the ATRA content of the formulations tested during the three-month storage is shown in Table 12, and a summary of ATRA-related impurities of the formulations tested during the three-month storage is shown in Table 13.

As shown in Tables 12 and 13, the ATRA content is more stable during the three months storage for formulations prepared according to the procedures described in Example 3 and 4 (that is, Formulations I and II) as compared to the formulations prepared according to the procedures described in Examples 6 and 7 (that is, Formulations III-VI). The ATRA contents in Formulations I and II were surprisingly stable during the three months storage at 25° C. The unexpected stability of Formulations I and II indicates that these formulations may be stored at room temperature rather than 5° C. during the product shelf life.

TABLE 12
Stability results based on ATRA content
Formulation I

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA (% | ATRA (% | ATRA (% | ATRA (% | ATRA (% | ATRA (% | ATRA (% |
| Position | w/w) | w/w) | w/w) | w/w) | w/w) | w/w) | w/w) | w/w) |
| Beginning (3 tubes) | 0.102 | 0.103 | 0.103 | 0.104 | 0.102 | 0.103 | 0.104 | 0.101 |
| Middle (3 tubes) | 0.102 | 0.102 | 0.103 | 0.104 | 0.101 | 0.102 | 0.103 | 0.101 |
| End (3 tubes) | 0.102 | 0.101 | 0.102 | 0.102 | 0.101 | 0.101 | 0.101 | 0.101 |
| Average (9 tubes) | 0.102 | 0.102 | 0.103 | 0.103 | 0.102 | 0.102 | 0.103 | 0.101 |
| % RSD (9 tubes) | 0.5 | 1.0 | 0.7 | 1.0 | 1.0 | 1.0 | 1.0 | 0.5 |
| % Initial Content | 100.0 | 99.9 | 100.7 | 101.3 | 99.7 | 100.2 | 100.6 | 99.0 |

Formulation II

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA (% | ATRA (% | ATRA (% | ATRA (% | ATRA (% | ATRA (% | ATRA (% |
| Position | w/w) | w/w) | w/w) | w/w) | w/w) | w/w) | w/w) | w/w) |
| Beginning (3 tubes) | 0.101 | 0.101 | 0.102 | 0.098 | 0.101 | 0.101 | 0.101 | 0.100 |
| Middle (3 tubes) | 0.099 | 0.102 | 0.099 | 0.098 | 0.101 | 0.100 | 0.100 | 0.099 |
| End (3 tubes) | 0.099 | 0.101 | 0.100 | 0.099 | 0.100 | 0.100 | 0.101 | 0.099 |
| Average (9 tubes) | 0.099 | 0.101 | 0.100 | 0.099 | 0.100 | 0.100 | 0.101 | 0.099 |
| % RSD (9 tubes) | 2.0 | 1.0 | 1.2 | 1.4 | 1.0 | 1.0 | 1.0 | 0.5 |
| % Initial Content | 100.0 | 101.9 | 101.0 | 99.1 | 100.9 | 101.0 | 101.2 | 99.9 |

Formulation III

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA (% | ATRA (% | ATRA (% | ATRA (% | ATRA (% | ATRA (% | ATRA (% |
| Position | w/w) | w/w) | w/w) | w/w) | w/w) | w/w) | w/w) | w/w) |
| Beginning (3 tubes) | 0.097 | 0.096 | 0.095 | 0.094 | — | 0.091 | 0.088 | — |
| Middle (3 tubes) | 0.097 | 0.095 | 0.095 | 0.095 | — | 0.091 | 0.038 | — |
| End (3 tubes) | 0.097 | 0.095 | 0.095 | 0.094 | — | 0.091 | 0.037 | — |
| Average (9 tubes) | 0.097 | 0.095 | 0.095 | 0.094 | — | 0.091 | 0.087 | — |
| % RSD (9 tubes) | 1.1 | 0.7 | 0.9 | 0.6 | — | 0.9 | 0.8 | — |
| % Initial Content | 100.0 | 98.4 | 98.3 | 97.5 | — | 93.8 | 90.2 | — |

Formulation IV

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA (% | ATRA (% | ATRA (% | ATRA (% | ATRA (% | ATRA (% | ATRA (% |
| Position | w/w) | w/w) | w/w) | w/w) | w/w) | w/w) | w/w) | w/w) |
| Average (3 samples) | 0.105 | — | 0.105 | — | 0.105 | 0.098 | 0.093 | 0.088 |
| % RSD (3 sarnples) | 0.6 | — | 1.1 | — | 0.5 | 0.6 | <0.1 | 0.7 |
| % Initial Content | 100.0 | — | 100 | — | 100 | 93.9 | 88.9 | 83.8 |

Formulation V

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA (% | ATRA (% | ATRA (% | ATRA (% | ATRA (% | ATRA (% | ATRA (% |
| Position | w/w) | w/w) | w/w) | w/w) | w/w) | w/w) | w/w) | w/w) |
| Average (3 samples) | 0.104 | — | 0.105 | — | 0.103 | 0.096 | 0.091 | 0.086 |
| % RSD (3 samples) | 1.0 | — | <0.1 | — | 0.6 | 0.6 | <0.1 | 0.7 |
| % Initial Content | 100.0 | — | 101.0 | — | 99.4 | 92.6 | 87.5 | 82.3 |

Formulation VI

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA | ATRA (% | ATRA (% | ATRA (% | ATRA (% | ATRA (% | ATRA (% |
| Position | w/w) | (% w/w) | w/w) | w/w) | w/w) | w/w) | w/w) | w/w) |
| Average (3 samples) | 0.108 | — | 0.101 | — | 0.103 | 0.099 | 0.099 | 0.093 |
| % RSD (3 samples) | 1.9 | — | 0.6 | — | 1.9 | 1.5 | 1.0 | 1.1 |
| % Initial Content | 100.0 | — | 93.8 | — | 95.4 | 92.0 | 91.7 | 86.1 |

Formulation VII

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | ATRA (% | ATRA | ATRA (% | ATRA (% | ATRA (% | ATRA (% | ATRA (% | ATRA (% |
| Position | w/w) | (% w/w) | w/w) | w/w) | w/w) | w/w) | w/w) | w/w) |
| Average (3 samples) | 0.109 | — | 0.108 | — | 0.109 | 0.105 | 0.103 | 0.101 |
| % RSD (3 samples) | 1.6 | — | 0.5 | — | 0.9 | 1.1 | 1.1 | 0.8 |
| % Initial Content | 100.0 | — | 99.4 | — | 100.0 | 96.0 | 94.8 | 92.4 |

TABLE 13
Stability results based on ATRA-related impurities:
Formulation I

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Beginning (3 tubes) | 0.556 | 0.625 | 0.634 | 0.619 | 0.649 | 0.752 | 0.806 | 0.867 |
| Middle (3 tubes) | 0.388 | 0.661 | 0.678 | 0.613 | 0.515 | 0.685 | 0.953 | 0.934 |
| End (3 tubes) | 0.414 | 0.664 | 0.644 | 0.583 | 0.680 | 0.702 | 0.823 | 0.864 |
| Average (9 tubes) | 0.453 | 0.650 | 0.652 | 0.605 | 0.615 | 0.713 | 0.868 | 0.888 |
| % RSD (9 tubes) | 20 | 7 | 6 | 7 | 14 | 7 | 14 | 5 |

Formulation II

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Beginning (3 tubes) | 0.586 | 0.472 | 0.447 | 0.396 | 0.600 | 0.778 | 0.638 | 0.818 |
| Middle (3 tubes) | 0.329 | 0.402 | 0.489 | 0.369 | 0.423 | 0.637 | 0.762 | 0.814 |
| End (3 tubes) | 0.361 | 0.436 | 0.521 | 0.351 | 0.642 | 0.650 | 0.660 | 0.822 |
| Average (9 tubes) | 0.426 | 0.437 | 0.476 | 0.372 | 0.555 | 0.638 | 0.687 | 0.618 |
| % RSD (9 tubes) | 30 | 16 | 15 | 12 | 22 | 13 | 11 | 9 |

Formulation III

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Beginning (3 tubes) | 0.736 | 0.832 | 0.778 | 1.004 | — | 1.614 | 1.929 | — |
| Middle (3 tubes) | 0.765 | 0.354 | 0.861 | 0.959 | — | 1.650 | 1.912 | — |
| End (3 tubes) | 0.767 | 0.329 | 0.835 | 0.980 | — | 1.584 | 1.868 | — |
| Average (9 tubes) | 0.756 | 0.833 | 0.325 | 0.931 | — | 1.616 | 1.903 | — |
| % RSD (9 tubes) | 7 | 3 | 8 | 6 | — | 3 | 3 | — |

Formulation IV

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Average (3 samples) | 1.801 | — | 0.746 | — | 0.780 | 1.835 | 2.623 | 2.765 |
| % RSD (3 samples) | 3 | — | 3 | — | 5 | 3 | 3 | 2 |

Formulation V

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Average (3 samples) | 1.860 | — | 1.070 | — | 0.850 | 1.421 | 2.157 | 2.122 |
| % RSD (3 samples) | 9 | — | 7 | — | 2 | 2 | 4 | 2 |

Formulation VI

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Average (3 samples) | 1.266 | — | 0.472 | — | 0.369 | 0.913 | 1.260 | 1.161 |
| % RSD (3 samples) | 14 | — | 7 | — | 8 | 9 | 9 | 4 |

Formulation VII

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | % Total | % Total | % Total | % Total | % Total | % Total | % Total | % Total |
| Position | Impurities | Impurities | Impurities | Impurities | Impurities | Impurities | Impurities | Impurities |
| Average (3 samples) | 0.936 | — | 0.607 | — | 0.494 | 1.114 | 1.597 | 1.462 |
| % RSD (3 samples) | 4 | — | 5 | — | 5 | 4 | 5 | 0 |

### BPO Stability Analysis

BPO was extracted from each of the formulations into THF and acetonitrile. The BPO content was determined in comparison with an external standard by HPLC method using Agilent 1200 HPLC system or equivalent. The HPLC conditions used are shown in Table 14.

TABLE 14
HPLC conditions for BPO content analysis

| Column | Zorbax Eclipse XDB-C18 4.6*150 mm 5 μm | | |
|---|---|---|---|
| Mobile phase | Eluent A - acetonitrile | | |
| | Eluent B - water | | |
| Gradient conditions | Time (min) | Eluent A (%) | Eluent B (%) |
| | Initial | 60 | 40 |
| | 8 | 60 | 40 |
| | 8.5 | 95 | 5 |
| | 11 | 95 | 5 |
| | 11.1 | 60 | 40 |
| | 16 | 60 | 40 |

| | |
|---|---|
| Flow rate | 1.1 ml/min |
| Detection | UV, wavelength 254 nm |
| Injection volume | 5 μL |
| Column temperature | 30° C. |

The BPO content in the sample was calculated using the formula:

$$\% BPO = \frac{Asample * V * Dsample * \% Pstd}{Rf * Wsample}$$

where:
Aₛₐₘₚₗₑ=BPO peak area arising from the Sample Preparation;
Dₛₐₘₚₗₑ=Sample dilution factor (4)
Wₛₐₘₚₗₑ=sample weight in mg;
Rf=averaged response factor (area/concentration) (average of five injections of first standard and one injection of second standard);
V=sample solution volume (50 ml);
P=purity of the standard (portion of BPO in the Standard)*(%) (* hydrous BPO Standard contains about 75% BPO)

The changes in BPO content during the three-month storage is summarized in Table 15.

TABLE 15
Summary of stability results based on BPO Content:
Formulation I

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 6.01 | 6.09 | 6.07 | 6.09 | 6.10 | 6.08 | 6.04 | 6.05 |
| % Difference | 0.3 | 0.3 | 0.2 | 0.3 | 0.2 | 0.0 | 0.3 | 0.7 |
| % Initial Content | 100.0 | 101.3 | 100.9 | 101.4 | 101.4 | 101.2 | 100.4 | 100.7 |

Formulation II

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 6.03 | 6.19 | 5.92 | 6.00 | 6.14 | 5.99 | 6.03 | 6.08 |
| % Difference | 1.5 | 1.1 | 0.0 | 1.0 | 0.5 | 0.5 | 0.2 | 0.7 |
| % Initial Content | 100.0 | 102.7 | 98.3 | 99.5 | 101.8 | 99.3 | 100.1 | 100.9 |

Formulation III

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 5.93 | 6.11 | 5.79 | 5.95 | — | 5.82 | 5.91 | — |
| % Difference | 0.8 | 2.0 | 0.7 | 0.3 | — | 0.2 | 2.0 | — |
| % Initial Content | 100.0 | 103.1 | 97.7 | 100.4 | — | 96.1 | 99.7 | — |

Formulation IV

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 6.13 | — | 6.29 | — | 6.33 | 6.24 | 6.22 | 6.19 |
| % Difference | 0.2 | — | 0.3 | — | 0.3 | 0.2 | 1.9 | 0.3 |
| % Initial Content | 100.0 | — | 102.7 | — | 103.3 | 101.8 | 101.6 | 101.1 |

Formulation V

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 5.83 | — | 5.92 | — | 5.93 | 5.99 | 5.96 | 5.87 |
| % Difference | 0.5 | — | 0.2 | — | 0.3 | <0.1 | 0.2 | 0.9 |
| % Initial Content | 100.0 | — | 100.7 | — | 100.9 | 102 | 101.4 | 99.8 |

Formulation VI

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 5.76 | — | 6.06 | — | 6.05 | 5.91 | 5.91 | 5.94 |
| % Difference | <0.1 | — | 0.3 | — | 1.7 | 0.2 | 0.5 | 0.5 |
| % Initial Content | 100.0 | — | 105.2 | — | 105.0 | 102.5 | 102.5 | 103.0 |

Formulation VII

| | | Freeze/ | 5° C. | | | 25° C. | | |
|---|---|---|---|---|---|---|---|---|
| | T0 | Thaw | 1 Month | 2 Months | 3 Months | 1 Month | 2 Months | 3 Months |
| | BPO (% | BPO (% | BPO (% | BPO (% | BPO (% | BPO (% | BPO (% | BPO (% |
| | w/w) | w/w) | w/w) | w/w) | w/w) | w/w) | w/w) | w/w) |
| Average (2) | 5.82 | — | 6.04 | — | 6.02 | 6.07 | 6.02 | 6.06 |
| % Difference | 0.7 | — | 0.5 | — | 0.8 | 0.8 | <0.1 | 0.2 |
| % Initial Content | 100.0 | — | 103.7 | — | 103.4 | 104.2 | 103.4 | 104.0 |

### Example 9. Viscosity of Formulations of Encapsulated ATRA and Encapsulated BPO

Formulations prepared according to the procedures described in Examples 3, 4, 6 and 7 were stored at 5° C. and 25° C./60% RH, respectively, for three months to study their changes in viscosity over time.

For the six formulations prepared according to the procedures described in Examples 4, 6 and 7 (Formulation I-VII), viscosity of each formulation was measured using Brookfield LVDV-H+Pro viscometer under the following conditions: spindle speed 1 rpm, total measuring time: 10 minutes, and temperature 30° C.±0.5° C. For Formulations II and III, spindle #63 (LV3) was used. For Formulations TV-VII, spindle # SC4-25 was used.

For the formulation prepared according to the procedures described in Example 3 (Formulation I), viscosity was measured using Brookfield LVDV-II+Pro viscometer equipped with helipath stand under the following conditions: spindle speed 2 rpm, measuring time interval: 30 seconds, total measuring time: 4 minutes, at room temperature (20° C.-25° C.).

The viscosities of the formulations tested during the three months storage are summarized in Table 16.

TABLE 16
Summary of viscosities during three-month storage

| | Formulation I | | Formulation II | | | | Formulation III | |
|---|---|---|---|---|---|---|---|---|
| Storage | 5° C. | 25° C. | 5° C. | | 25° C. | | 5° C. | 25° C. |
| time | Viscosity | Viscosity | Viscosity | | Viscosity | | Viscosity | Viscosity |
| (month) | (cps) | (cps) | (cps) | | (cps) | | (cps) | (cps) |
| 0 | 105200 | 105200 | 54600 | | 54600 | | 77400 | 77400 |
| 1 | 148400 | 154200 | 53880 | | 48240 | | 73920 | 65160 |
| 2 | 179400 | 168600 | 51480 | | 36960 | | 64080 | 55440 |
| 3 | 163800 | 166600 | 51240 | | 44760 | | 77400 | 77400 |
| | Formulation IV | | Formulation V | | Formulation VI | | Formulation VII | |
| Storage | 5° C. | 25° C. | 5° C. | 5° C. | 5° C. | 25° C. | 5° C. | 25° C. |
| time | Viscosity | Viscosity | Viscosity | Viscosity | Viscosity | Viscosity | Viscosity | Viscosity |
| (month) | (cps) | (cps) | (cps) | (cps) | (cps) | (cps) | (cps) | (cps) |
| 0 | 415200 | 415200 | 271600 | 271600 | 122800 | 122800 | 109900 | 109900 |
| 1 | 467500 | 457900 | 251500 | 167000 | 123800 | 104100 | 90720 | 68160 |
| 2 | — | 446400 | — | 285100 | — | 90240 | — | 51840 |
| 3 | 474700 | 479500 | 367600 | 390700 | 130500 | 72480 | 88800 | 41280 |

### Example 10. Microbial Growth in Formulations of Encapsulated ATRA and Encapsulated BPO

Formulations prepared according to the procedures described in Examples 3 and 4 (Formulations I and II) were stored at 25° C./60% RH for three months to study the microbial growth over time. The results of microbial growth are summarized in Table 17. It was surprising to find that no significant yeast or mold growth in Formulation I (that is, the formulation prepared according to the procedures described in Example 3), which further indicates that this formulation may be stored at room temperature rather than 5° C. during the product shelf life.

TABLE 17
Microbial growth at 25° C. in E-ATRA and E-BPO formulations

| Storage | | | |
|---|---|---|---|
| time | | | |
| (month) | | Formulation I | Formulation II |
| 0 | Total Aerobic Count | <20 CFU/gram | <20 CFU/gram |
| | Yeasts and Molds | <20 CFU/gram | <20 CFU/gram |
| | Specified Microorganisms | Absence of | Absence of |
| | per 1 g (Staphylococcus | S. aureus and | S. aureus and |
| | aureus and Pseudomonas | P. aeruginosa | P. aeruginosa |
| | aeruginosa) | | |
| 1 | Total Aerobic Count | — | — |
| | Yeasts and Molds | — | — |
| | Specified Microorganisms | — | — |
| | per 1 g | | |
| 2 | Total Aerobic Count | — | — |
| | Yeasts and Molds | — | — |
| | Specified Microorganisms | — | — |
| | per 1 g | | |
| 3 | Total Aerobic Count | <20 CFU/gram | <20 CFU/gram |
| | Yeasts and Molds | <20 CFU/gram | 20 CFU/gram |
| | Specified Microorganisms | Absence of | Absence of |
| | per 1 g | S. aureus and | S. aureus and |
| | | P. aeruginosa | P. aeruginosa |

### Example 11. Antimicrobial Effectiveness Testing of Formulation (I)

Antimicrobial Effectiveness Testing (AET) of Formulation (I) was conducted in accordance with USP 51 at a storage condition of 40° C. for one month. The results are summarized in Table 18 and show that Formulation (I) meets criteria of USP 51.

TABLE 18
AET of Formulation (I) at 40° C.

| | | Log reduction | | Log reduction |
|---|---|---|---|---|
| | 14 | from initial | 28 | from the |
| | day | calculated count | day | 14 day's count |
| | cfu | at 14 day | cfu | at 28 days |
| Bacteria | | | | |
| S. aureus | <10 | >4.9 | <10 | 0 |
| E. Coli | <10 | >4.6 | <10 | 0 |
| P. aeruginosa | <10 | >4.9 | <10 | 0 |
| Yeast and Molds | | | | |
| C. albicans | <10 | >5.0 | <10 | 0 |
| A. niger | <10 | >4.1 | <10 | 0 |

### Example 12. Microbial Limits Testing of Formulation (I) and Formulation (II)

Microbial Limits Testing (MLT) of Formulation (I) and Formulation (II) was conducted in accordance with USP 61 at a storage condition of 25° C. for six months. Briefly, a formulation is considered to meet the criteria of USP 61 when testing under these conditions results in a total bacteria count of not more than 200 cfu and a total yeast and mold count of no more than 20 cfu. Both Formulation (I) and Formulation (II) met this criteria; however the total yeast and mold count for Formulation (II) was 20 cfu. This result indicates a potential for other samples or variants of Formulation (I) and Formulation (II) to not meet the criteria of USP 61, as measured by Microbial Limits Testing (MLT) after storage of the composition at a storage condition of 25° C. for six months, and/or a potential to not meet the criteria of USP 51, as measured by Antimicrobial Effectiveness Testing (AET) after storage of the composition at a storage condition of 40° C. for one month.

### Example 13. Preparation of Formulation of Encapsulated ATRA (0.1%) and Encapsulated BPO (6%) in Emulsion with Antimicrobial Stabilizing Agent (Formulation VIII)

Formulation (VIII) was made in a manner similar to that described above in Example 3 for Formulation (I) except that antimicrobials (methyl paraben and imidazolidinyl urea) were included, as follows:

### Preparation of Encapsulated ATRA (3.06% E-ATRA Water Suspension)

### a) Oil Phase

8.62 grams of Butylated hydroxyl toluene (BHT) and 45.9 grams of all-trans retinoic acid (ATRA) were mixed in 129.3 grams of Squalane. 86.16 grams of Tetroethoxysilane (TEOS) were added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled tretinoin in oil was aliquoted out and then heated to 60° C. 9.0 grams of Beeswax were added and melted in the oil phase.

### b) Water Phase

3.3 grams CTAC (Cetrimonium Chloride) were dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) was added to the water phase and homogenized at 4000 rpm for 1 minute. 17.9 grams of sodium silicate extra pure solution (28%) were added to the emulsion. The pH of the emulsion was adjusted to 4.0 using HCl 5N solution. Water was added to complete the total weight of the mixture to 650 grams. The suspension was then stirred for 17 hours at 25° C. for the TEOS hydrolysis to be completed. The composition of the final encapsulated ATRA water suspension product is shown in Table 19.

TABLE 19
Composition of the encapsulated ATRA 3.06% water suspension

| | % of pure ingredient in the |
|---|---|
| Ingredient | suspension |
| Beeswax | 1.15 |
| Squalane | 8.62 |
| TEOS | 5.74 |
| ATRA | 3.06 |
| Cetrimonium Chloride | 0.15 |
| Sodium hydroxide | 0.74 |
| Hydrochloric acid | 0.40 |
| Butylated hydroxytoluene | 0.57 |
| Sterile Water for Irrigation | 79.56 |

### Preparation of Encapsulated BPO (15% E-BPO Water Suspension)

### (a) Preparation of Benzoyl Peroxide Solution and Acid Cocktail

A benzoyl peroxide (BPO) solution was prepared by mixing 125.67 grams of CTAC CT-429 (Cetrimonium Chloride 30%), 3008 grams of hydrous benzoyl peroxide, and 5200 grams water under high shear. The solution was homogenized for 60 minutes at 33° C. (no more than 45° C.), and then the pH of the solution was adjusted to 7.0 using sodium hydroxide solution (20%).

An acid cocktail was prepared using 493 grams Hydrochloric acid (37%), 98 grams anhydrous Citric Acid, 147 grams Lactic Acid (90%), and 794 grams water.

### (b) Coating Cycle

The coating cycle was started by adding 38 grams sodium silicate solution extra pure (28%) to the benzoyl peroxide solution prepared in step (a) under high shear, followed by adding the acid cocktail prepared in step (a) to adjust the pH to be lower than 6.8, and followed by adding 57 grams PDAC (3%) solution to the mixture. The cycle was repeated 50 times while the mixture was stirred under high shear for 17 hours. After the 50 cycles, the pH of the mixture was adjusted to 5.0 using the acid cocktail, and water was added to complete the total weight of the mixture to 15 kilograms. The composition of the final BPO water suspension product is shown in Table 20.

TABLE 20
Composition of the encapsulated BPO 15% water suspension

| | % of ingredient in the |
|---|---|
| Ingredient | suspension |
| Polyquarternium-7 | 0.53 |
| Hydrochloric Acid | 0.85 |
| Citric Acid, Anhydrous | 0.50 |
| Lactic Acid | 0.63 |
| Silicon Dioxide | 3.42 |
| Sodium hydroxide | 0.006 |
| Cetrimonium Chloride | 0.25 |
| Hydrous Benzoyl Peroxide | 15.00 |
| Sterile Water for Irrigation | 78.80 |

### Preparation of Formulation (VIII): Encapsulated ATRA (0.1%) and Encapsulated BPO (6%) in Emulsion with Antimicrobials

Oil Phase: 720.0 grams of Cyclomethicone 5-N, 540.0 grams of Cetyl Alcohol, 360.0 grams of Polyoxyl 100 Stearate, and 540.0 grams of Mono- and Di-glycerides were mixed at 70° C.

Water phase: 18.0 grams of Ethylendiaminetetraacetate Disodium salt were dissolved in 6500 grams of water. 720.0 grams of glycerin (99.5%), 45.0 grams of methyl paraben, and 45.0 grams of imidazolidinyl urea were added to the solution. After the solution was, mixed at 2800 rpm for 10 minutes, 72.0 grams of Carbopol 980 NF were added and the resulting mixture was homogenized at 3300 rpm for 10 minutes to ensure that all materials completely melted and dissolved. The solution was heated to 70° C. with continuous mixing, 76.5 grams of sodium hydroxide (20%) were then added and the mixture was stirred under high shear for 10 minutes at no less than 70° C.

The oil phase was added to the water phase under high shear at 70° C., and the resulting emulsion was homogenized at 3300 rpm for 10 minutes. 72.0 grams of Citric Acid and 7152 grams of encapsulated BPO 15% water suspension made as described in Example 2 were mixed. The resulting mixture was added to the emulsion at 65° C. and mixed at 1400 rpm for 10 minutes. The emulsion was cooled to 35° C. and the pH of the emulsion was adjusted to 3.5 using HCl 5N solution. After 588.2 grams of encapsulated ATRA 3.06% water suspension made as described in Example 1 were added, the emulsion was stirred at 1400 rpm for 10 minutes. HCl 5N was added to adjust the pH to 3.4-3.7, and then water was added until the total weight of the emulsion reached 18 kilograms. The composition of the formulation prepared in this example is shown in Table 21.

TABLE 21
Composition of Formulation VIII

| | % of pure ingredient in the |
|---|---|
| Ingredient | composition |
| Polyquarternium-7 | 0.21 |
| Hydrochloric Acid | 0.51 |
| Citric Acid, Anhydrous | 0.60 |
| Lactic Acid | 0.25 |
| Silicon Dioxide | 1.44 |
| Sodium hydroxide | 0.09 |
| Cetrimonium Chloride | 0.11 |
| Hydrous Benzoyl Peroxide | 6.00 |
| Beeswax | 0.04 |
| Squalane | 0.28 |
| Ethanol (Alcohol) | 0.14 |
| ATRA | 0.10 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Mono- and Di-Glycerides | 3.00 |
| Edetate Disodium | 0.10 |
| Carbopol 980 | 0.40 |
| Methyl Paraben (antimicrobial) | 0.25 |
| Imidazolidinyl urea (antimicrobial) | 0.25 |
| Sterile Water for Irrigation | 73.21 |

### Example 14. Stability of Formulation (VIII) Under Various Storage Conditions

Bulk viscosity, ATRA level and BPO level were measured for samples of compositions prepared as described above in Example 13 for Formulation (VIII) after storage under the indicated conditions. The results are shown in Tables 22-24, respectively.

TABLE 22
Bulk Viscosity Stability of Formulation (VIII)

| | | | |
|---|---|---|---|
| Initial | 1 Month | 2 Months | 3 Months |

5° C. Storage

| | | | | |
|---|---|---|---|---|
| Bulk Viscosity | 335800 | — | — | 428400 |

25° C. Storage

| | | | | |
|---|---|---|---|---|
| Bulk Viscosity | 335800 | 288800 | — | 258800 |

30° C. Storage

| | | | | |
|---|---|---|---|---|
| Bulk Viscosity | 335800 | 336600 | 301000 | 283600 |

TABLE 23
ATRA Stability of Formulation (VIII)

| | | | |
|---|---|---|---|
| Initial | 1 Month | 2 Months | 3 Months |

5° C. Storage

| | | | | |
|---|---|---|---|---|
| ATRA (% w/w) - | 0.098 | — | — | 0.100 |
| Average (3) | | | | |
| ATRA (% Initial Content) - | 100.0 | — | — | 102.0 |
| Average (3) | | | | |

25° C. Storage

| | | | | |
|---|---|---|---|---|
| ATRA (% w/w) - | 0.098 | 0.099 | — | 0.094 |
| Average (3) | | | | |
| ATRA (% Initial Content) - | 100.0 | 101.0 | — | 95.9 |
| Average (3) | | | | |

38° C. Storage

| | | | | |
|---|---|---|---|---|
| ATRA (% w/w) - | 0.098 | 0.092 | 0.092 | 0.088 |
| Average (3) | | | | |
| ATRA (% Initial Content) - | 100.0 | 93.9 | 93.9 | 89.8 |
| Average (3) | | | | |

TABLE 24
BPO Stability of Formulation ( VIII)

| | | | |
|---|---|---|---|
| Initial | 1 Month | 2 Months | 3 Months |

5° C. Storage

| | | | | |
|---|---|---|---|---|
| BPO (% w/w) - | 5.80 | — | — | 6.00 |
| Average (2) | | | | |
| BPO (% Initial Content) - | 100.0 | — | — | 103.4 |
| Average (2) | | | | |

25° C. Storage

| | | | | |
|---|---|---|---|---|
| BPO (% w/w) - | 5.80 | 5.96 | — | 5.82 |
| Average (2) | | | | |
| BPO (% Initial Content) - | 100.0 | 102.8 | — | 100.3 |
| Average (2) | | | | |

38° C. Storage

| | | | | |
|---|---|---|---|---|
| BPO (% w/w) - | 5.80 | 5.90 | 6.02 | 5.81 |
| Average (2) | | | | |
| BPO (% Initial Content) - | 100.0 | 101.7 | 103.8 | 100.2 |
| Average (2) | | | | |

### Examples 15-33. Effect of Antimicrobial Selection on ATRA Level for Variants of Formulation (VIII)

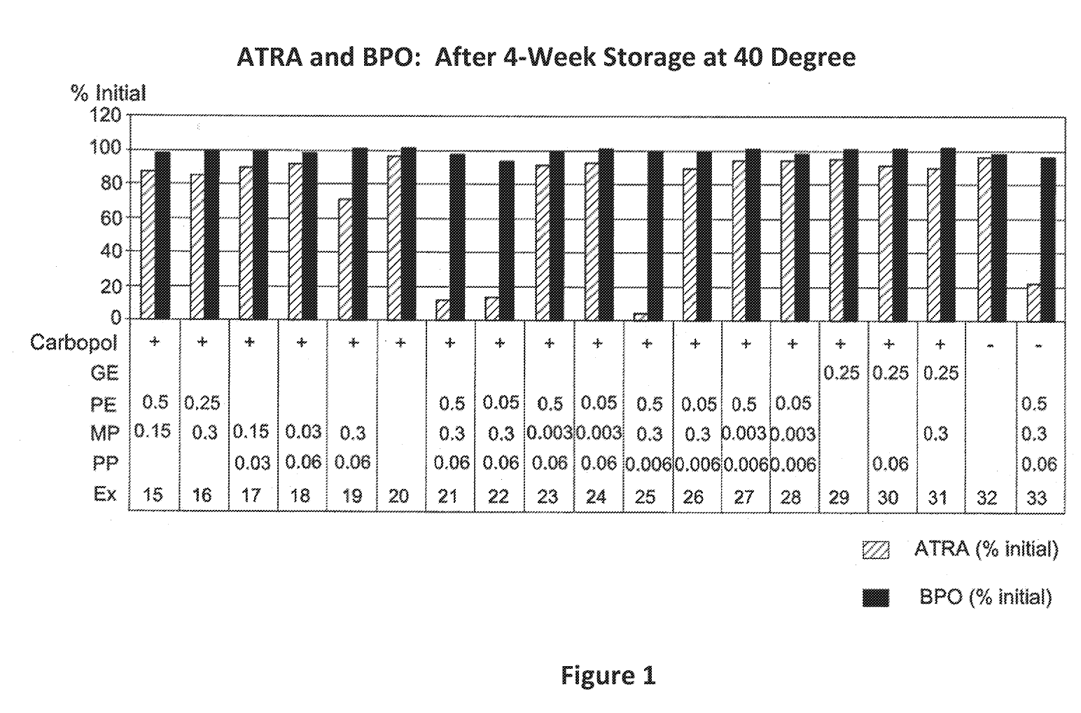

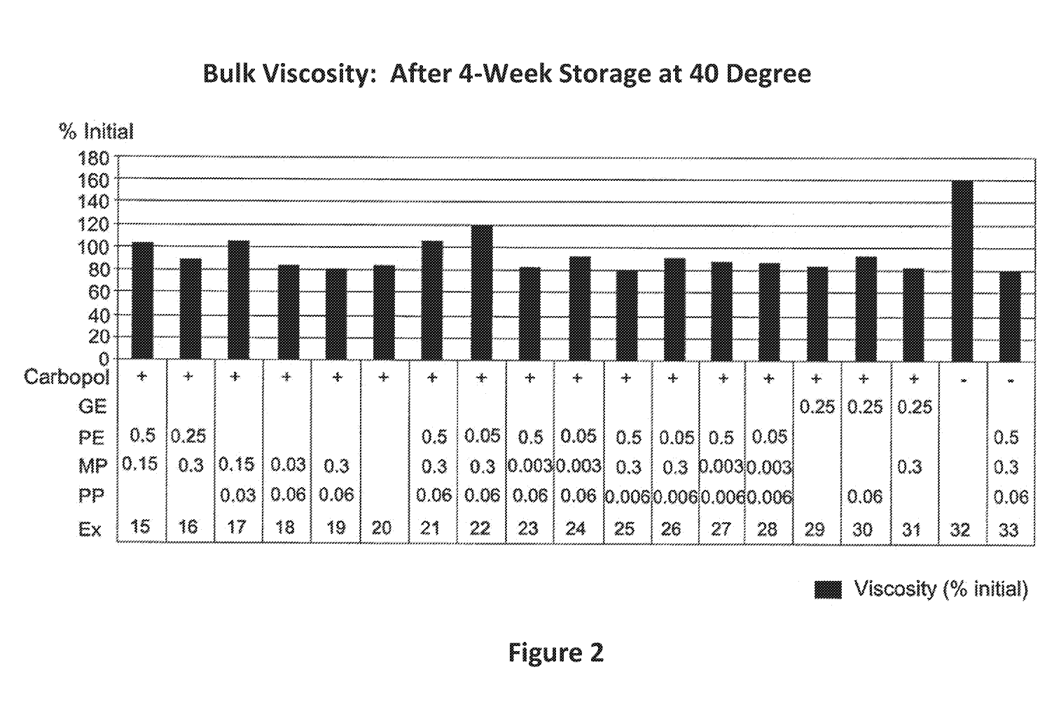

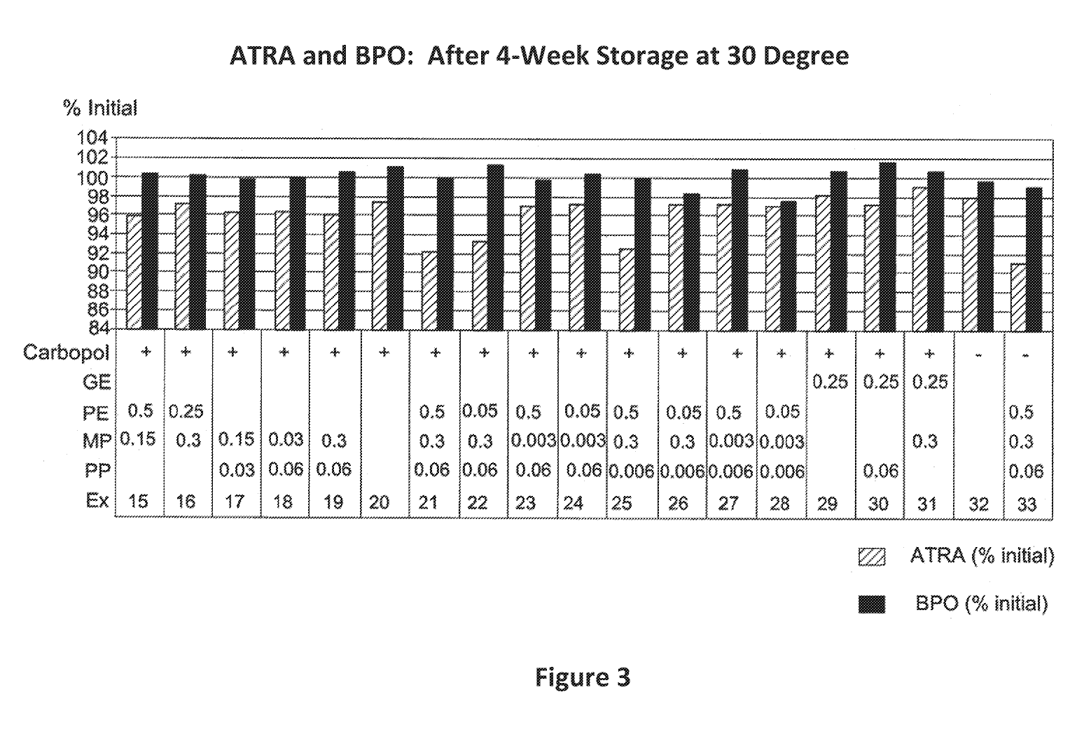

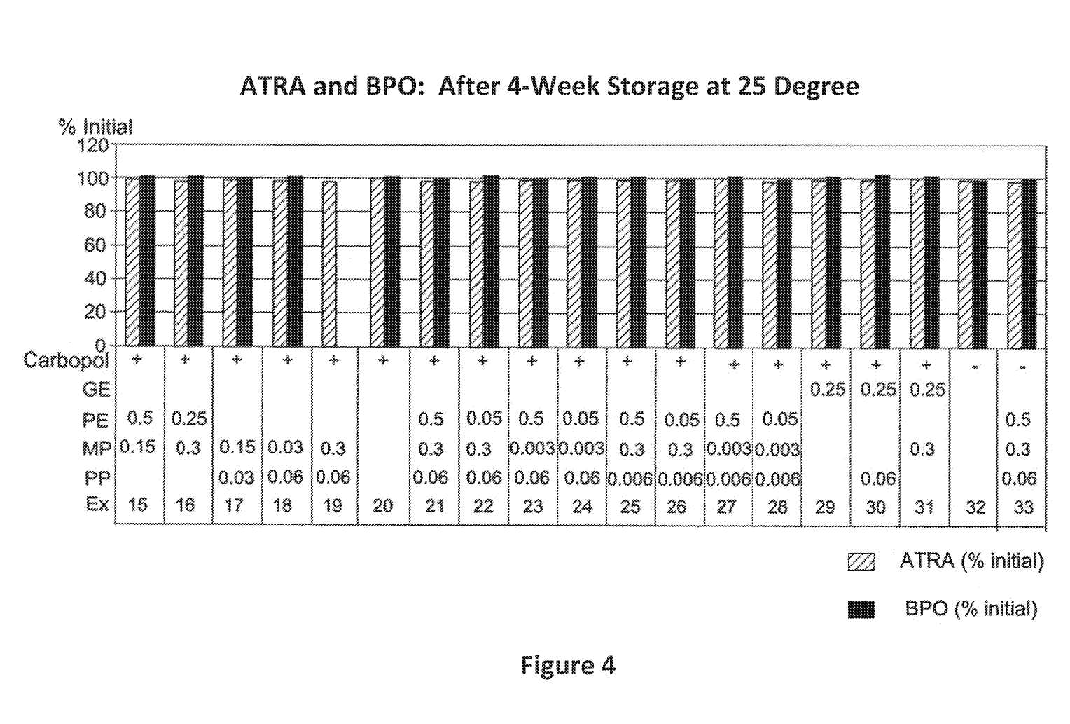

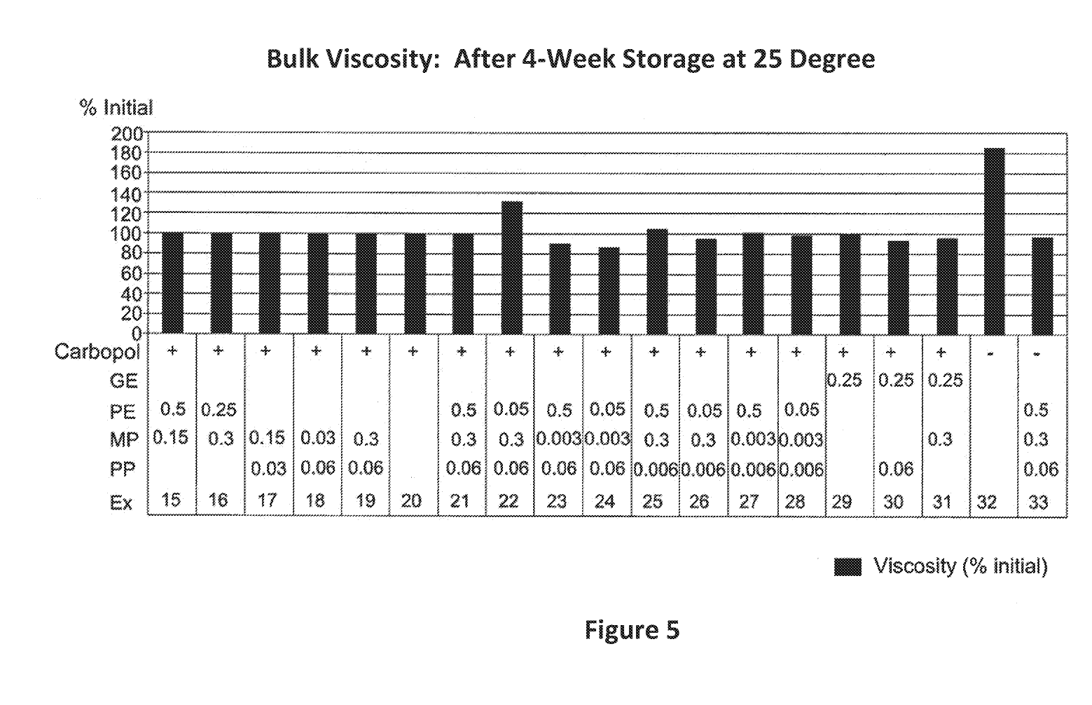

A series of ATRA/BPO formulations (shown in FIGS. 1 through 5) were prepared in a manner similar to that described above in Example 13 for Formulation (VIII) except that the Carbopol content and type and amount of antimicrobial were varied. Measurements of initial bulk viscosity, initial ATRA content and initial BPO content were made. Samples of the ATRA/BPO formulations were then stored for four weeks at 40° C., 30° C. or 25° C. Measurements of bulk viscosity, ATRA content and BPO content were made after storage, and are illustrated in FIGS. 1-5 in terms of the percentage of the initial values. The amount of antimicrobials (wt. % of methylparaben (MP), propylparaben (PP), phenoxyethanol (PE), and/or imidazolidinyl urea (GE)) and the presence (+) or absence (−) of 0.4% Carbopol in each of the formulations are also shown in FIGS. 1-5.

### Example 34. Stability Study of a Non-Encapsulated ATRA/BPO Composition

Tables 12-15 of this disclosure details the surprisingly long term high stability of ATRA/BPO compositions of this invention.

This Example provides data on the stability of a non-encapsulated ATRA/BPO composition.
The composition comprising non-encapsulated BPO 2.5% and non-encapsulated tretinoin 0.1% was found to be stable for at least 2 months at 25° C./65% RH and 40° C./75% RH.

### Examples 35-38. Compositions for the Dual Chamber Dispenser and Two Separate Dispensers

### Example 35. Preparation of Water-Dispersed ATRA Composition (3.06% ATRA Water Suspension)

### a) Oil Phase

8.62 grams of Butylated hydroxyl toluene (BHT) and 45.9 grams of all-trans retinoic acid (ATRA) were mixed in 129.3 grams of Squalane. and the resulted mixture was milled at 5000 rpm in a ball mill for 10 minutes with an upper propeller mixer at a speed of 250 rpm for 7 minutes, followed by 400 rpm for 3 minutes. 140.4 grams of milled tretinoin in oil was aliquoted out and then heated to 60° C. 9.0 grams of Beeswax were added and melted in the oil phase.

### b) Water Phase

3.3 grams CTAC (Cetrimonium Chloride) were dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) was added to the water phase and homogenized at 4000 rpm for 1 minute. 17.9 grams of silicon dioxide were added to the emulsion. The pH of the emulsion was adjusted to 4.0 using HCl 5N solution. Water was added to complete the total weight of the mixture to 650 grams. The suspension was then stirred for 2 hours at 25° C. The composition of the final ATRA water suspension product is shown in Table 25.

TABLE 25
Composition of ATRA 3.06% water suspension

| | % of pure ingredient in the |
|---|---|
| Ingredient | suspension |
| Beeswax | 1.15 |
| Squalane | 8.62 |
| Silicon dioxide | 0.81 |
| ATRA | 3.06 |
| Cetrimonium Chloride | 0.15 |
| Hydrochloric acid | 0.40 |
| Butylated hydroxytoluene | 0.57 |
| Sterile Water for Irrigation | 85.24 |

### Example 36. Preparation of BPO 15% Water Suspension

### a) Preparation of Benzoyl Peroxide Suspension and Acid Cocktail

A benzoyl peroxide (BPO) suspension was prepared by mixing 125.67 grams of CTAC CT-429 (Cetrimonium Chloride 30%), 3008 grams of hydrous benzoyl peroxide, and 5200 g water under high shear. The suspension was homogenized for 60 minutes at 33° C. (no more than 45° C.), and then the pH of the solution was adjusted to 7.0 using sodium hydroxide solution (20%). An acid cocktail was prepared using 493 gram Hydrochloric acid (37%), 98 gram anhydrous Citric Acid, 147 gram Lactic Acid (90%), and 794 grams water.

### b) Suspension Stabilization

Stabilization of the suspension was done by adding 532 grams of silicon dioxide to the benzoyl peroxide suspension prepared in step a) under high shear, followed by adding 855 grams PDAC (10%/) solution to the mixture. The mixture was stirred under high shear for 2 hours. The pH of the mixture was adjusted to 5.0 using acid cocktail and water was added to complete the total weight of the mixture to 15 kilograms. The composition of the final BPO water suspension product is shown in Table 26.

TABLE 26
Composition of BPO 15% water suspension

| | % of ingredient in the |
|---|---|
| Ingredient | suspension |
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Hydrous Benzoyl Peroxide | 15.00 |
| Sterile Water for Irrigation | 78.83 |

### Example 37. Preparation of Dual Chamber Formulation of ATRA 0.1% Cream and BPO 6% Cream

Step I: preparation of BPO 6% cream

Oil Phase: 720.0 of grams Cyclomethicone 5-N, 540.0 of grams Cetyl Alcohol, 360.0 grams Polyoxyl 100 Stearate, and 540.0 grams of Glyceryl Monosterate were mixed at 70° C.

Water phase: 18.0 grams of Ethylendiaminetetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 7152 grams of BPO 15% water suspension made as described in Example 36 were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms. The composition of the formulation prepared in this example is shown in Table 27.

TABLE 27
Composition of BPO 6% cream

| | % of pure ingredient in the |
|---|---|
| Ingredient | composition |
| Polyquarternium-7 | 0.21 |
| Hydrochloric Acid | 0.51 |
| Citric Acid, Anhydrous | 0.58 |
| Lactic Acid | 0.25 |
| Silicon Dioxide | 1.39 |
| Cetrimonium Chloride | 0.10 |
| Hydrous Benzoyl Peroxide | 6.00 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | 74.46 |

Step II: preparation of ATRA 0.1% cream

Oil Phase: 720.0 of grams Cyclomethicone 5-N, 540.0 of grams Cetyl Alcohol, 360.0 grams Polyoxyl 100 Stearate, and 540.0 grams of Glyceryl Monosterate were mixed at 70° C.

Water phase: 18.0 grams of Ethylendiaminetetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 588.2 grams of ATRA 3.06% water suspension made as described in Example 35 were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms. The composition of the formulation prepared in this example is shown in Table 28.

TABLE 28
Composition of ATRA 0.1% cream

| | % of pure ingredient in the |
|---|---|
| Ingredient | composition |
| Hydrochloric Acid | 0.41 |
| Citric Acid, Anhydrous | 0.44 |
| Silicon Dioxide | 0.05 |
| Cetrimonium Chloride | 0.01 |
| Beeswax | 0.04 |
| Squalane | 0.28 |
| ATRA | 0.10 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | 82.15 |

Step III: preparation of dual chamber product

### Dual Chamber Dispensing for the Topical Application of ATRA/BPO Compositions

A dual chamber pump was purchased from Lablabo®, solo Twinbag®, 2×30 ml volume, single actuator with 2 nozzles. The pump has 2 compartments. BPO 6% cream, as prepared in step I, was filled in the 1^{st} compartment. ATRA 0.1% cream, as prepared in step II, was filled in the 2^{nd} compartment. When the patient presses the actuator 0.25 gram of each cream is dispensed from each nozzle. A mixture is obtained, and about 0.5 gram of the BPO/ATRA combination product comprising 3% BPO and 0.05% ATRA is applied on the skin of the patient.

### Example 38. Two Dispenser Formulation of ATRA 0.1% Cream and BPO 6% Cream

A first dispenser (such as a 30 ml tube) of 30 ml volume is filled with the BPO 6% cream, as prepared in step I of Example 37. A second dispenser (such as a 30 ml tube) of 30 ml volume is filled with ATRA 0.1% cream, as prepared in step H of Example 37. The patient dispenses about 0.25 g of composition from each dispenser and mixes the two compositions on the skin, thus administering about 0.5 g of the combination product comprising 3% BPO and 0.05% ATRA on the skin of the patient.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treatment of acne in a patient in need thereof, comprising the concomitant topical administration once daily for a period of up to 3 months of a stable composition, comprising from about 2% w/w to about 10% w/w of solid particulate benzoyl peroxide (BPO) and from about 0.01% w/w to about 0.1% w/w of solid particulate all trans retinoic acid (ATRA),
wherein 90% of the solid particles of ATRA are in the range of 5-20 microns,
wherein the side-effects of the two active agents BPO and ATRA are medically acceptable and
wherein the therapeutic effect of BPO and ATRA is superior to the effect of each active agent administered alone.

2. The method of claim 1, wherein none of the two active agents BPO and ATRA are encapsulated.

3. The method of claim 1, wherein one of the two active agents BPO and ATRA is encapsulated either partially or entirely.

4. The method of claim 1, wherein both active agents BPO and ATRA are encapsulated either partially or entirely.

5. The method of claim 1, wherein said stable composition comprising a plurality of first core-shell microcapsules comprising a first core that comprises BPO and a first shell that comprises a first inorganic polymer; and a plurality of second core-shell microcapsules comprising a second core that comprises ATRA and a second shell that comprises a second inorganic polymer; wherein said composition is an oil in water emulsion comprising a polyoxylstearate and a glycerylstearate, wherein the ratio of said polyoxylstearate to said glycerylstearate is in the range of 0.1:10 to 10:0.1.

6. The method of claim 1, wherein said stable composition comprising an initial concentration of 3% w/w BPO and an initial concentration of 0.05% w/w ATRA and wherein the composition maintains the amount of BPO at a level of at least about 2.7% by weight based on the total weight of the composition, as measured after storage of the composition at a storage condition selected from the group consisting of 40° C. for two months, 30° C. for three months, 25° C. for six months and 5° C. for two years and maintains the amount of ATRA at a level of at least about 0.045% by weight based on the total weight of the composition, as measured after storage of the composition at a storage condition selected from the group consisting of 40° C. for two months, 30° C. for three months, 25° C. for six months and 5° C. for two years; (ii) maintain the microbial count of the composition at a level that meets the criteria of USP 51, as measured by Antimicrobial Effectiveness Testing (AET) after storage of the composition at a storage condition of 40° C. for one month; and maintains the bulk viscosity of said composition at about one million cps or less and within a range of about 70% to about 130% of the initial bulk viscosity, as measured after storage of the composition at 25° C. for three months.

7. The method of claim 1 wherein the concentration of ATRA in the stable composition is from about 0.04% w/w to about 0.1% w/w, preferably about 0.1% w/w, more preferably about 0.08% w/w and even more preferably about 0.05% w/w or about 0.04% w/w.

8. The method of claim 1 wherein the concentration of BPO is from about 2% w/w to about 10% w/w, preferably from about 2.5% w/w to about 5% w/w, preferably about 3.75% w/w or about 6.28% w/w and more preferably about 3% w/w.

9. The method of claim 1, wherein the concentration of BPO is 3% w/w and the concentration of ATRA is from about 0.05% w/w to about 0.1% w/w.

10. The method of claim 1, wherein the stable composition is administered to a patient in need thereof for up to 2 weeks, up to 1 month, preferably up to 2 months and more preferably up to 3 months.

11. The method of claim 1, wherein the concomitant administration is carried out by once daily topical administration to a patient in need thereof of a single composition comprising both BPO and ATRA.

12. The method of claim 1, wherein the concomitant administration is carried out by topical administration of a first composition comprising from about 2% w/w to about 10% w/w BPO, and a second composition comprising from about 0.1% w/w to about 0.2% w/w ATRA, concomitantly administered once daily from a dual chamber dispenser or from two separate dispensers and mixed before applying on the skin of a patient in need thereof, thus administering a combination product comprising from about 1% w/w to about 5% w/w BPO and from about 0.05% w/w to about 0.1% w/w ATRA on the skin of said patient.

13. The method of claim 1, wherein the concomitant administration is carried out by topical administration of a first composition comprising 6% w/w BPO, and a second composition comprising 0.1% w/w ATRA, concomitantly administered once daily for a period of up to 3 months from a dual chamber dispenser or from two separate dispensers and mixed before applying on the skin of a patient in need thereof, thus administering a combination product comprising 3% BPO and 0.05% ATRA on the skin of said patient.

14. The method of claim 12, wherein the first composition comprises a plurality of first core-shell microcapsules comprising a first core that comprises BPO and a first shell that comprises a first inorganic polymer and the second composition comprises a plurality of second core-shell microcapsules comprising a second core that comprises ATRA and a second shell that comprises a second inorganic polymer; wherein said composition is an oil in water emulsion comprising a polyoxylstearate and a glycerylstearate wherein the ratio of said polyoxylstearate to said glycerylstearate is in the range of 0.1:10 to 10:0.1.

15. A dual chamber dispenser with a single actuator and two nozzles, having a first chamber charged with a first composition comprising from about 2% w/w to about 10% w/w BPO, and a second chamber charged with a second composition comprising from about 0.1% w/w to about 0.2% w/w ATRA, wherein the two compositions are mixed before applying on the skin of a patient in need thereof, thus administering a combination product comprising from about 1% w/w to about 5% w/w BPO and from about 0.05% w/w to about 0.1% w/w ATRA on the skin of said patient.

16. The dual chamber dispenser of claim 15, wherein the first composition comprises 6% w/w BPO, and the second composition comprises 0.1% w/w ATRA, wherein the two compositions are mixed before applying on the skin of a patient in need thereof, thus administering a combination product comprising 3% BPO and 0.05% ATRA on the skin of said patient.

17. A regimen of administration comprising the method of claim 1, comprising daily administration to a patient in need thereof of a stable composition of claim 1 comprising from about 2% w/w to about 10% w/w of solid particulate benzoyl peroxide (BPO) and from about 0.01% w/w to about 0.1% w/w of solid particulate all trans retinoic acid (ATRA) for up to 2 weeks, up to 1 month, preferably up to 2 months and more preferably up to 3 months.

18. A regimen of administration comprising daily administration to a patient in need thereof of a first composition comprising from about 2% w/w to about 10% w/w BPO, and a second composition comprising from about 0.1% w/w to about 0.2% w/w ATRA, wherein the two compositions are concomitantly administered once daily from a dual chamber dispenser or from two separate dispensers and mixed before applying on the skin of a patient in need thereof for up to 2 weeks, up to 1 month, preferably up to 2 months and more preferably up to 3 months.
